(19) United States
(12) Patent Application Publication
(10) Pub. No.: US 2012/0082688 A1
(43) Pub. Date: Apr. 5, 2012

(54) In Vitro Generation of Myeloid Derived Suppressor Cells

(75) Inventors: Shu-Hsia Chen, New York, NY (US); Zuping Zhou, New York, NY (US); Ping-Ying Pan, New York, NY (US)

(73) Assignee: MOUNT SINAI SCHOOL OF MEDICINE, New York, NY (US)

(21) Appl. No.: 13/131,450

(22) PCT Filed: Nov. 25, 2009
(86) PCT No.: PCT/US09/65981

§ 371 Date: Dec. 15, 2011

Related U.S. Application Data
(60) Provisional application No. 61/118,273, filed on Nov. 26, 2008.

(51) Int. Cl. A61K 35/32; A61P 37/06; A61P 37/02; C12N 5/0775

(52) U.S. Cl. 424/184.1; 435/377; 435/372

(57) ABSTRACT

The invention relates to methods of isolating, culturing, and differentiating myeloid derived suppressor cells (MD-SCs) from embryonic stem (ES) cells and hematopoietic stem cells (HSCs). In certain embodiments, the invention relates to methods and compositions for producing MDSCs from ES cells and HSCs using a combination of factors including macrophage colony-stimulating factor (M-CSF).

### CROSS-REFERENCE TO RELATED APPLICATIONS

Priority is claimed to U.S. provisional application Ser. No. 61/118,273, filed Nov. 26, 2008. The contents of this priority application are hereby incorporated into the present disclosure by reference in their entirety.

### FIELD OF THE INVENTION

The invention relates to methods of isolating, culturing, and differentiating myeloid derived suppressor cells (MDSCs) from embryonic stem cells (ES) and hematopoietic stem cells (HSCs). In certain embodiments, the invention relates to methods and compositions for producing MDSCs from ES cells and HSCs using M-CSF.

### BACKGROUND

Myeloid-derived suppressor cells (MDSCs) are a heterogeneous population of immature myeloid cells with immunoregulatory activity. (Serafini P, Borrello I, Bronte V. Myeloid suppressor cells in cancer: recruitment, phenotype, properties, and mechanisms of immune suppression. Semin Cancer Biol. 2006; 16:53-65). These cells, defined in mice by surface expression of CD11b and Gr-1, can function to suppress antigen-specific and non-specific T cell responses via diverse mechanisms, e.g. production of nitric oxide (NO), reactive oxygen species (ROS), expression of arginase (Rodriguez P C, Hernandez C P, Quiceno D, et al. Arginase I in myeloid suppressor cells is induced by COX-2 in lung carcinoma. J Exp Med. 2005; 202:931-939, and Rodriguez P C, Quiceno D G, Zabaleta J, et al. Arginase I production in the tumor microenvironment by mature myeloid cells inhibits T-cell receptor expression and antigen-specific T-cell responses. Cancer Res. 2004; 64:5839-5849), inducible nitric oxide synthetase (iNOS), and/or secretion of IL-10 and TGF-β. There is also evidence that MDSCs play a role in various pathological conditions. In animal tumor models (Melani C, Chiodoni C, Forni G, Colombo M P. Myeloid cell expansion elicited by the progression of spontaneous mammary carcinomas in c-erbB-2 transgenic BALB/c mice suppresses immune reactivity. Blood. 2003; 102:2138-2145) and cancer patients (Young M R, Lathers D M. Myeloid progenitor cells mediate immune suppression in patients with head and neck cancers. Int J Immunopharmacol. 1999; 21:241-252, and Almand B, Clark J I, Nikitina E, et al. Increased production of immature myeloid cells in cancer patients: a mechanism of immunosuppression in cancer. J Immunol. 2001; 166:678-689), MDSCs, induced by tumor-derived factors, accumulate in large number in the blood, bone marrow, spleen, and tumor masses, mediating T cell tolerance, and thus leading to tumor escape and progression (Shojaei F, Wu X, Zhong C, et al. Bv8 regulates myeloid-cell-dependent tumour angiogenesis. Nature. 2007; 450:825-831, Tu S, Bhagat G, Cui G, et al. Overexpression of interleukin-1beta induces gastric inflammation and cancer and mobilizes myeloid-derived suppressor cells in mice. Cancer Cell. 2008; 14:408-419, Yang L, DeBusk L M, Fukuda K, et al. Expansion of myeloid immune suppressor Gr+CD11b+ cells in tumor-bearing host directly promotes tumor angiogenesis. Cancer Cell. 2004; 6:409-421, and Yang L, Huang J, Ren X, et al. Abrogation of TGF beta signaling in mammary carcinomas recruits Gr-1+CD11b+ myeloid cells that promote metastasis. Cancer Cell. 2008; 13:23-35). MDSCs, together with tumor-associated macrophages as well as T regulatory cells present in the tumor niche, are now considered as the major factors responsible for the limited effectiveness or failure of cancer vaccines and other immunotherapies (Pollard J W. Tumour-educated macrophages promote tumour progression and metastasis. Nat Rev Cancer. 2004; 4:71-78, Condeelis J, Pollard J W. Macrophages: obligate partners for tumor cell migration, invasion, and metastasis. Cell. 2006; 124:263-266, Curiel T J, Coukos G, Zou L, et al. Specific recruitment of regulatory T cells in ovarian carcinoma fosters immune privilege and predicts reduced survival. Nat Med. 2004; 10:942-949, Shojaei F, Wu X, Malik A K, et al. Tumor refractoriness to anti-VEGF treatment is mediated by CD11b+Gr1+ myeloid cells. Nat Biotechnol. 2007; 25:911-920, Shojaei F, Ferrara N. Refractoriness to antivascular endothelial growth factor treatment: role of myeloid cells. Cancer Res. 2008; 68:5501-5504, and Sinha P, Clements V K, Bunt S K, Albelda S M, Ostrand-Rosenberg S. Cross-talk between myeloid-derived suppressor cells and macrophages subverts tumor immunity toward a type 2 response. J Immunol. 2007; 179:977-983).

Potential clinical application of MDSCs has been hampered by the lack of cells due to the paucity of MDSCs in vivo. While several immortalized myeloid suppressor cell lines have been reported, their characterization and stability are unknown. (See for example, Apolloni, E. V., et al., J. Immunol. 165:6723-6730, 2000). Furthermore, immortalized cell lines were generated by transducing vmyc and v-raf oncogenes, which represent a safety concern when used in vivo.

Thus, needs exist in the art to isolate, culture, sustain, propagate, and differentiate MDSCs, particularly from sources that are relatively accessible in order to develop cell types suitable for a variety of uses.

### SUMMARY OF THE INVENTION

In certain embodiments, the present invention provides a method of preparing an isolated myeloid derived suppressor cell (MDSC) comprising: a) contacting an embryonic stem (ES) cell with an effective amount of kit ligand (KL) (stem cell factor), vascular endothelial growth factor (VEGF), FMS-like tyrosine kinase 3 (Flt3L), thrombopoietin (TPO), and macrophage colony-stimulating factor (M-CSF); and b) culturing said ES under conditions suitable for propagation of said cell, thereby obtaining a preparation of an isolated MDSC.

In certain embodiments, the method further comprises cryopreservation of said MDSC. In yet additional embodiments, the ES cell is a mammalian ES cell. In certain embodiments, the ES cell is a human ES cell. In yet additional embodiments, the isolated MDSC expresses at least one of the cell surface markers selected from the group consisting of CD33, CD115, F4/80, Ly-6C, CD11b, Gr-1, VEGF receptor, CD40 and IL-4R.

In certain embodiments, the present invention provides a method of preparing an isolated myeloid derived suppressor cell (MDSC) comprising: a) contacting a hematopoietic stem cell (HSC) with an effective amount of kit ligand (KL) (stem cell factor), vascular endothelial growth factor (VEGF), FMS-like tyrosine kinase 3 (Flt3L), thrombopoietin (TPO), and macrophage colony-stimulating factor (M-CSF); and b) culturing said HSC under conditions suitable for propagation of said cell, thereby obtaining a preparation of an isolated MDSC.

In certain embodiments, the method further comprises cryopreservation of said MDSC. In yet additional embodiments, the HSC is a mammalian HSC. In yet additional embodiments, the HSC is a human HSC. In yet additional embodiments, the isolated MDSC expresses at least one of the cell surface markers selected from the group consisting of CD33, CD115, VEGF receptor, F4/80, Ly-6C, CD11b, Gr-1, CD40 and IL-4R. In other embodiments, the isolated MDSC derived from a human ES cell or human HSC expresses at least one of the cell surface markers selected from the group consisting of CD11b, CD33, CD15, and CD16. In yet other embodiments, the isolated MDSC expresses CD11b and CD33. In still other embodiments, the isolated MDSC expresses CD11b and Gr-1.

In yet additional embodiments, the invention provides an isolated MDSC obtained by any of the methods described herein.

In certain embodiments, the present invention provides a method of treating a disorder amenable to cell-based treatment in a mammal, comprising administering a pharmaceutically effective amount of the isolated MDSC obtained by any of the methods described herein, to a mammal in need thereof. In certain embodiments, the disorder is selected from the group consisting of graft-versus-host disease (GVHD) and an autoimmune disorder.

### BRIEF DESCRIPTION OF THE DRAWINGS

FIGS. 1A-F are chromatograms and graphs showing directed differentiation of Hoxb4 ES cells into myeloid-derived suppressor cells.

FIG. 1A is a chromatogram showing the kinetics of myeloid suppressor cell development.

FIG. 1B is a chromatogram showing that cells incubated without induction of hoxB4 expression did not grow well, with nearly 90 percent dead cells.

FIG. 1C is a chromatogram illustrating the opposite roles of M-CSF and GM-CSF in the generation of ES cell-derived CD115⁺ cells.

FIG. 1D is a graph illustrating dose-dependent effect of M-CSF in the generation of ES cell-derived CD115⁺ cells.

FIG. 1E is a graph showing the efficiency of myeloid-derived suppressor cell production. FIG. 1F is a schematic showing a three-step differentiation strategy to generate myeloid suppressor cells from HoxB4-ESs.

FIGS. 2A-D are graphs showing the potent suppressive capacity of ES-MDSCs. For FIGS. 2A-D the Y-axis is the proliferation CPM counts determined by incorporation of [3H]-thymidine. The X-axis is the serial dilution of the sorted CD115⁺ and CD115⁻ cell number with a fixed number of splenocytes.

FIG. 2A shows inhibition of polycolonally stimulated T cell proliferation by ES cell-derived CD115⁺ cells.

FIG. 2B shows inhibition of alloantigen-stimulated T cell proliferation by ES cell-derived CD115⁺ cells.

FIG. 2C shows suppression of T cell proliferation by both CD115+Ly-6C⁺ and CD115+Ly-6C populations.

FIG. 2D shows a comparison of suppressive activity of ES cell-derived CD115⁺ cells versus purified CD115⁺ cells isolated from tumor-bearing mice (TD-CD115⁺).

For FIGS. 2A-D the Y-axis is the proliferation CPM counts determined by incorporation of ³H-thymidine. The X-axis is the serial dilution of the sorted CD115⁺ and CD115⁻ cell number with a fixed number of splenocytes.

FIGS. 3A-C are chromatograms, graphs, and RT-PCR results showing various pathways mediating suppressor function of ES-MDSCs.

FIG. 3A is a chromatogram showing Treg development induced by ES-MDSCs.

FIG. 3B is a graph showing NO production by ES-MDSCs.

FIG. 3C is a photograph of a gel of RT-PCR results showing expression of iNOS, arginase 1, IL-10 and TGF-β by ES-MDSCs.

FIGS. 4A-F are graphs and photographs of cultures showing morphological, phenotypic, and developmental characterization of ES-MDSCs. Cells used in FIG. 4A-F were obtained on day 10 after differentiation on OP9 in M3 (IL-6, IL-3, IL-β, βFGF, erythropoietin (EPO), TPO, VEGF, Flt-3L, M-CSF, GM-CSF.

FIG. 4A is a photograph showing the morphology of ES cell-derived MDSCs.

FIG. 4B are graphs showing surface phenotypes of different subsets differentiated from Hoxb4 ESs. Cells recovered were directly stained with a panel of surface markers and analyzed by flow cytometry.

FIG. 4C are chromatograms showing progression of CD115 population indicating that the majority of the originally seeded CD115⁺Ly-6C⁻ (FIG. 4C, upper panel) and CD115⁺Ly-6C⁺ (FIG. 4C, lower panel) cells progressed gradually to become a population expressing CD115 and lower level of Ly-6C (these cells were immature macrophages as evaluated by Giemsa staining).

FIG. 4D shows that the CD115⁺Ly-6C⁻ cell cultures also gave rise to two additional minor populations, CD115⁺Ly-6C⁻ cells and CD115⁻Ly-6C⁺ (granulocyte), lineage potentials of which were entirely dependent on GM-CSF (FIG. 4D).

FIG. 4E are graphs showing the colony forming activity of FACS-sorted CD115+Ly-6C⁻ and CD115+Ly-6C⁺ populations in methylcellulose. CD115+Ly-6C⁻ cells, which had higher clonogenic activity than CD115+Ly-6C⁺ cells (upper graph), gave rise to M, GM, G colonies, whereas the CD115+Ly-6C⁺ population generated only M colonies (lower graph). M, macrophage; GM, macrophage and granulocyte; G, granulocyte.

FIG. 4F is a photograph is May-Giemsa stained cytospins of pooled colonies developed from CD115+Ly-6C⁻ cells (upper photo, arrows and arrowheads indicated macrophages and granulocytes, respectively) and CD115+Ly-6C⁺ cells (lower photo). Original magnification: 400×.

FIGS. 5A-C are graphs and photographs of cultures illustrating the prevention of allo-HSCT-associated GVHD by ES-MDSCs.

FIG. 5A is a graph showing the survival curve of recipient mice.

FIG. 5B is a graph showing changes of mean body weight of each group of mice (▪, n=6; ▴, n=3˜10; , n=9˜11 mice).

FIG. 5C are photographs of H&E-stained sections of target tissues harvested on day 23 from indicated groups.

FIGS. 6A-C are chromatograms and graphs showing the generation of myeloid-derived suppressor cells from marrow hematopoietic stem/progenitor cells.

FIG. 6A is a schematic of chromatograms showing in vitro differentiation of marrow hematopoietic stem/progenitor cells into myeloid-derived suppressor cells (HS-MDSC).

FIG. 6B is a graph showing the comparison of suppressive activity between HS-MDSC and TD-MDSC.

FIG. 6C shows chromatograms showing enhanced Treg induction by HS-MDSCs.

FIG. 6D is a graph showing splenocyte proliferation in cocultures with MDSCs isolated from wild-type (Wt), IL-10-deficient, inos-deficient, or IL-4 deficient mice and in the presence of anti-CD3/anti-CD28. The Y-axis is the proliferation CPM counts determined by incorporation of [3H]-thymidine. The X-axis is the serial dilution of the MDSCs with a fixed number of splenocytes.

FIG. 6E shows splenocyte proliferation in cocultures with HSC-MDSCs or TD-MDSCs in the presence of anti-CD3/anti-CD28 and the indicated concentration of the iNOS inhibitor L-NMMA (“L”) and/or the arginase inhibitor NOHA (“N”). The Y-axis is the proliferation CPM counts determined by incorporation of [3H]-thymidine. The X-axis is the indicated coculture treated with the indicated inhibitor(s).

### DETAILED DESCRIPTION

The present invention encompasses methods for preparing an isolated myeloid derived suppressor cell (MDSC) by a) contacting an embryonic stem (ES) cell with an effective amount of kit ligand (KL) (stem cell factor), vascular endothelial growth factor (VEGF), FMS-like tyrosine kinase 3 (Flt3L), thrombopoietin (TPO), and macrophage colony-stimulating factor (M-CSF); and b) culturing the ES cell under conditions suitable for propagation of the cell, to obtain a preparation of an isolated MDSC.

ES cells possess numerous attractive attributes such as pluripotency, infinite self-renewal, and feasibility of genetic manipulation (e.g., gene transduction), making them an ideal source for producing large quantities of a specific cell type or tissue.

Thus, in certain embodiments, the present methods encompass an efficient system in which functionally active MDSCs can be generated from ES cells (“ES-MDSCs”), and the methods are also applicable to derivation of MDSCs from hematopoietic stem (HSCs or HS) cells. The in vitro ES cell-derived MDSCs obtained by the methods described herein were comparable with those isolated from tumor-bearing mice in morphology, phenotypes, functional activities and development, but had slightly stronger suppressive activity and significantly enhanced ability to induce Treg development. The in vitro differentiation of ES or HSCs into MDSCs provides an unlimited source for immunotherapeutics and for investigating the differentiation and accumulation of MDSCs.

In certain embodiments, the present invention encompasses methods to generate functionally active MDSCs. In one exemplification, functionally active MDSCs can be efficiently generated from ES cells using a three-stage differentiation procedure. In other aspects, functionally active MDSCs can be generated from HSCs using similar differentiation methods. ES cell-derived MDSCs encompass two homogenous populations: namely CD115⁺Ly-6C⁻ and CD115⁺Ly-6C⁺ cells, which in the context of morphology, phenotype, functional gene profile and development, resemble, respectively, the granulocyte/macrophage progenitors (GMPs) and the monocytic CD115⁺Gr1⁺F4/80⁺ cells (TD-MDSCs), a major component of MDSCs previously identified in tumor-bearing mice. ES-MDSCs exhibit robust suppression against T cell proliferation in vitro. Impressively, in comparison to TD-MDSCs, ES-MDSCs display slightly stronger suppressive activity and significantly enhanced ability to induce Treg development, two functional features also shared by HS-MDSCs. Furthermore, adoptive transfer of ES-MDSCs can effectively prevent allo-reactive T cell-mediated lethal graft-versus-host disease (GVHD), leading to nearly 82% long-term survival of treated mice. The in vitro generation of MDSCs represents a substantial advancement in the exploitation of potential clinical application of MDSCs. The in vitro generation of MDSCs will also provide an excellent alternative platform for studying the differentiation and biological function of MDSCs in pathologic settings.

In additional embodiments, the invention encompasses preparing isolated myeloid derived suppressor cells (MDSC) by a) contacting an HSC with an effective amount of KL (stem cell factor), VEGF, Flt3L, and TPO (thrombopoietin), and macrophage colony-stimulating factor (M-CSF); and b) culturing the HSC under conditions suitable for propagation of the cell, thereby obtaining a preparation of an isolated MDSC.

M-CSF was originally described by Price, L. K., et al., “The predominant form of secreted colony stimulating factor-1 is a proteoglycan,” J. Biol. Chem. 267 (4), 2190-2199 (1992), and has been characterized and various transcript variants can be found in GenBank. Human M-CSF, also called CSF-1 (for colony stimulating factor variant 1) has been characterized and has GenBank reference No. NM_{—}000757 (SEQ ID NO:1); the protein sequence has GenBank reference N0. NP_{—}000748 (SEQ ID NO:2). The mouse M-CSF sequences have also been characterized. The mouse nucleotide sequence for M-CSF also called CSF-1 (for colony stimulating factor variant 1) has GenBank reference No. NM_{—}007778 (SEQ ID NO:3); the protein sequence has GenBank reference No. NP_{—}031804 (SEQ ID NO:4).

Any suitable form of M-CSF can be utilized in the methods of the present invention, including purified M-CSF and recombinantly produced M-CSF. Additionally, variants, mutants, or fragments of M-CSF can be utilized as long as they are active and exhibit desired characteristics including stimulating ES cells to differentiate into MDSCs.

In accordance with the present invention there may be employed conventional molecular biology, microbiology, protein expression and purification, antibody, and recombinant DNA techniques within the skill of the art. Such techniques are explained fully in the literature. See, e.g., Sambrook et al. (2001) Molecular Cloning: A Laboratory Manual. 3^{rd} ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.; Nucleic Acid Hybridization, Hames & Higgins eds. (1985); Transcription And Translation, Hames & Higgins, eds. (1984); Animal Cell Culture Freshney, ed. (1986); Immobilized Cells And Enzymes, IRL Press (1986); Perbal, A Practical Guide To Molecular Cloning (1984); and Harlow and Lane. Antibodies: A Laboratory Manual (Cold Spring Harbor Laboratory Press: 1988).

### DEFINITIONS

The following definitions are provided for clarity and illustrative purposes only, and are not intended to limit the scope of the invention.

As used herein, the term “antigen presenting cell” refers to a cell that has the ability to present peptide or lipid antigen on surface major histocompatibility complex (MHC) molecules.

The term “growth factor” can be a naturally occurring, endogenous or exogenous protein, or recombinant protein, capable of stimulating cellular proliferation and/or cellular differentiation.

A procedure for “ex vivo expansion” of hematopoietic stem and progenitor cells (HSCs) is described in U.S. Pat. No. 5,199,942. Briefly, the term means a method comprising: (1) collecting CD34⁺ hematopoietic stem and progenitor cells from a patient from peripheral blood harvest or bone marrow explants; and (2) expanding such cells ex vivo. In addition to the cellular growth factors described in U.S. Pat. No. 5,199,942, other factors such as IL-1, IL-3, c-kit ligand, can be used.

As used herein, the term “gene transfer” refers to the transfer of genetic material to an organism.

The term “gene therapy” refers to the insertion of genes into an individual's cells and/or tissues to treat a disease. In certain embodiments, a mammal or patient may be administered an effective amount of a desired plasmid or viral vector containing the nucleic acid sequence to treat a disease. An effective amount of a viral vector or plasmid is defined herein as an amount of the viral vector or plasmid that, upon administration to a patient or mammal, results in the expression of an effective amount of the desired gene for treating the disease.

### EXPRESSION CONSTRUCT

“Expression construct” refers to a nucleic acid sequence comprising a target nucleic acid sequence or sequences whose expression is desired, operatively associated with expression control sequence elements which provide for the proper transcription and translation of the target nucleic acid sequence(s) within the chosen host cells. Such sequence elements may include a promoter and a polyadenylation signal. The “expression construct” may further comprise “vector sequences”. “Vector sequences” refer to any of several nucleic acid sequences established in the art which have utility in the recombinant DNA technologies of the invention to facilitate the cloning and propagation of the expression constructs including (but not limited to) plasmids, cosmids, phage vectors, viral vectors, and yeast artificial chromosomes.

Expression constructs of the present invention may comprise vector sequences that facilitate the cloning and propagation of the expression constructs. A large number of vectors, including plasmid and fungal vectors, have been described for replication and/or expression in a variety of eukaryotic and prokaryotic host cells. Standard vectors useful in the current invention are well known in the art and include (but are not limited to) plasmids, cosmids, phage vectors, viral vectors, and yeast artificial chromosomes. The vector sequences may contain a replication origin for propagation in E. coli; the SV40 origin of replication; an ampicillin, neomycin, or puromycin resistance gene for selection in host cells; and/or genes (e.g., dihydrofolate reductase gene) that amplify the dominant selectable marker plus the gene of interest.

### EXPRESS AND EXPRESSION

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “transfection” means the introduction of a foreign nucleic acid into a cell. The term “transformation” means the introduction of a “foreign” (i.e. extrinsic or extracellular) gene, DNA or RNA sequence to a cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cells genetic machinery. The gene or sequence may include nonfunctional sequences or sequences with no known function. A host cell that receives and expresses introduced DNA or RNA has been “transformed” and is a “transformant” or a “clone”. The DNA or RNA introduced to a host cell can come from any source, including cells of the same genus or species as the host cell, or cells of a different genus or species.

### EXPRESSION SYSTEM

The term “expression system” means a host cell and compatible vector under suitable conditions, e.g., for the expression of a protein coded for by foreign DNA carried by the vector and introduced to the host cell.

### GENE OR STRUCTURAL GENE

The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription.

A coding sequence is “under the control of” or “operatively associated with” expression control sequences in a cell when RNA polymerase transcribes the coding sequence into RNA, particularly mRNA, which is then trans-RNA spliced (if it contains introns) and translated into the protein encoded by the coding sequence.

The term “expression control sequence” refers to a promoter and any enhancer or suppression elements that combine to regulate the transcription of a coding sequence. In a preferred embodiment, the element is an origin of replication.

### HETEROLOGOUS

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. For example, the present invention includes chimeric DNA molecules that comprise a DNA sequence and a heterologous DNA sequence which is not part of the DNA sequence. A heterologous expression regulatory element is such an element that is operatively associated with a different gene than the one it is operatively associated with in nature. In the context of the present invention, a gene encoding a protein of interest is heterologous to the vector DNA in which it is inserted for cloning or expression, and it is heterologous to a host cell containing such a vector, in which it is expressed. In certain embodiments, heterologous is used to describe a cell that is transferred from one individual to another individual, and is therefore, not isolated from the recipient of the transferred cell.

### HOMOLOGOUS

The term “homologous” as used in the art commonly refers to the relationship between nucleic acid molecules or proteins that possess a “common evolutionary origin,” including nucleic acid molecules or proteins within superfamilies (e.g., the immunoglobulin superfamily) and nucleic acid molecules or proteins from different species (Reeck et al., Cell 1987; 50: 667). Such nucleic acid molecules or proteins have sequence homology, as reflected by their sequence similarity, whether in terms of substantial percent similarity or the presence of specific residues or motifs at conserved positions.

### HOST CELL

The term “host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells can further be used for screening or other assays that are described infra. Host cells may be cultured in vitro or one or more cells in a non-human animal (e.g., a transgenic animal or a transiently transfected animal). Suitable host cells include but are not limited to Streptomyces species and E. coli.

### TREATING OR TREATMENT

“Treating” or “treatment” of a state, disorder or condition includes:
(1) preventing or delaying the appearance of clinical or sub-clinical symptoms of the state, disorder or condition developing in a mammal that may be afflicted with or predisposed to the state, disorder or condition but does not yet experience or display clinical or subclinical symptoms of the state, disorder or condition; or
(2) inhibiting the state, disorder or condition, i.e., arresting, reducing or delaying the development of the disease or a relapse thereof (in case of maintenance treatment) or at least one clinical or sub-clinical symptom thereof; or
(3) relieving the disease, i.e., causing regression of the state, disorder or condition or at least one of its clinical or sub-clinical symptoms.

The benefit to a subject to be treated is either statistically significant or at least perceptible to the patient or to the physician.

### PATIENT OR SUBJECT

“Patient” or “subject” refers to mammals and includes human and veterinary subjects.

### THERAPEUTICALLY EFFECTIVE AMOUNT

A “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

### ABOUT OR APPROXIMATELY

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless otherwise stated, the term ‘about’ means within an acceptable error range for the particular value.

### DOSAGE

The dosage of the therapeutic formulation will vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level.

### CARRIER

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

### ISOLATED

As used herein, the term “isolated” means that the referenced material is removed from the environment in which it is normally found. Thus, an isolated biological material can be free of cellular components, i.e., components of the cells in which the material is found or produced. Isolated nucleic acid molecules include, for example, a PCR product, an isolated mRNA, a cDNA, or a restriction fragment. Isolated nucleic acid molecules also include, for example, sequences inserted into plasmids, cosmids, artificial chromosomes, and the like. An isolated nucleic acid molecule is preferably excised from the genome in which it may be found, and more preferably is no longer joined to non-regulatory sequences, non-coding sequences, or to other genes located upstream or downstream of the nucleic acid molecule when found within the genome. An isolated protein may be associated with other proteins or nucleic acids, or both, with which it associates in the cell, or with cellular membranes if it is a membrane-associated protein.

### MUTANT

As used herein, the terms “mutant” and “mutation” refer to any detectable change in genetic material (e.g., DNA) or any process, mechanism, or result of such a change. This includes gene mutations, in which the structure (e.g., DNA sequence) of a gene is altered, any gene or DNA arising from any mutation process, and any expression product (e.g., protein or enzyme) expressed by a modified gene or DNA sequence. As used herein, the term “mutating” refers to a process of creating a mutant or mutation.

### NUCLEIC ACID HYBRIDIZATION

The term “nucleic acid hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions. Stringency of hybridization is determined, e.g., by (i) the temperature at which hybridization and/or washing is performed, and (ii) the ionic strength and (iii) concentration of denaturants such as formamide of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (i.e., will not prevent formation of an anti-parallel hybrid). See Molecular Biology of the Cell, Alberts et al., 3rd ed., New York and London: Garland Publ., 1994, Ch. 7.

Typically, hybridization of two strands at high stringency requires that the sequences exhibit a high degree of complementarity over an extended portion of their length. Examples of high stringency conditions include: hybridization to filter-bound DNA in 0.5 M NaHPO₄, 7% SDS, 1 mM EDTA at 65° C., followed by washing in 0.1×SSC/0.1% SDS at 68° C. (where 1×SSC is 0.15M NaCl, 0.15M Na citrate) or for oligonucleotide molecules washing in 6×SSC/0.5% sodium pyrophosphate at about 37° C. (for 14 nucleotide-long oligos), at about 48° C. (for about 17 nucleotide-long oligos), at about 55° C. (for 20 nucleotide-long oligos), and at about 60° C. (for 23 nucleotide-long oligos)). Accordingly, the term “high stringency hybridization” refers to a combination of solvent and temperature where two strands will pair to form a “hybrid” helix only if their nucleotide sequences are almost perfectly complementary (see Molecular Biology of the Cell, Alberts et al., 3rd ed., New York and London: Garland Publ., 1994, Ch. 7).

Conditions of intermediate or moderate stringency (such as, for example, an aqueous solution of 2×SSC at 65° C.; alternatively, for example, hybridization to filter-bound DNA in 0.5 M NaHPO₄, 7% SDS, 1 mM EDTA at 65° C., and washing in 0.2×SSC/0.1% SDS at 42° C.) and low stringency (such as, for example, an aqueous solution of 2×SSC at 55° C.), require correspondingly less overall complementarity for hybridization to occur between two sequences. Specific temperature and salt conditions for any given stringency hybridization reaction depend on the concentration of the target DNA and length and base composition of the probe, and are normally determined empirically in preliminary experiments, which are routine (see Southern, J. Mol. Biol. 1975; 98: 503; Sambrook et al., Molecular Cloning: A Laboratory Manual, 2nd ed., vol. 2, ch. 9.50, CSH Laboratory Press, 1989; Ausubel et al. (eds.), 1989, Current Protocols in Molecular Biology, Vol. I, Green Publishing Associates, Inc., and John Wiley & Sons, Inc., New York, at p. 2.10.3).

As used herein, the term “standard hybridization conditions” refers to hybridization conditions that allow hybridization of sequences having at least 75% sequence identity. According to a specific embodiment, hybridization conditions of higher stringency may be used to allow hybridization of only sequences having at least 80% sequence identity, at least 90% sequence identity, at least 95% sequence identity, or at least 99% sequence identity.

Nucleic acid molecules that “hybridize” to any desired nucleic acids of the present invention may be of any length. In one embodiment, such nucleic acid molecules are at least 10, at least 15, at least 20, at least 30, at least 40, at least 50, and at least 70 nucleotides in length. In another embodiment, nucleic acid molecules that hybridize are of about the same length as the particular desired nucleic acid.

### NUCLEIC ACID MOLECULE

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

### ORTHOLOGS

As used herein, the term “orthologs” refers to genes in different species that apparently evolved from a common ancestral gene by speciation. Normally, orthologs retain the same function through the course of evolution. Identification of orthologs can provide reliable prediction of gene function in newly sequenced genomes. Sequence comparison algorithms that can be used to identify orthologs include without limitation BLAST, FASTA, DNA Strider, and the GCG pileup program. Orthologs often have high sequence similarity. The present invention encompasses all orthologs of the desired protein.

### OPERATIVELY ASSOCIATED

By “operatively associated with” is meant that a target nucleic acid sequence and one or more expression control sequences (e.g., promoters) are physically linked so as to permit expression of the polypeptide encoded by the target nucleic acid sequence within a host cell.

### PERCENT SEQUENCE SIMILARITY OR PERCENT SEQUENCE IDENTITY

The terms “percent (%) sequence similarity”, “percent (%) sequence identity”, and the like, generally refer to the degree of identity or correspondence between different nucleotide sequences of nucleic acid molecules or amino acid sequences of proteins that may or may not share a common evolutionary origin (see Reeck et al., supra). Sequence identity can be determined using any of a number of publicly available sequence comparison algorithms, such as BLAST, FASTA, DNA Strider, GCG (Genetics Computer Group, Program Manual for the GCG Package, Version 7, Madison, Wis.), etc.

To determine the percent identity between two amino acid sequences or two nucleic acid molecules, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are, or are about, of the same length. The percent identity between two sequences can be determined using techniques similar to those described below, with or without allowing gaps. In calculating percent sequence identity, typically exact matches are counted.

The determination of percent identity between two sequences can be accomplished using a mathematical algorithm. A non-limiting example of a mathematical algorithm utilized for the comparison of two sequences is the algorithm of Karlin and Altschul, Proc. Natl. Acad. Sci. USA 1990, 87:2264, modified as in Karlin and Altschul, Proc. Natl. Acad. Sci. USA 1993, 90:5873-5877. Such an algorithm is incorporated into the NBLAST and XBLAST programs of Altschul et al., J. Mol. Biol. 1990; 215: 403. BLAST nucleotide searches can be performed with the NBLAST program, score=100, wordlength=12, to obtain nucleotide sequences homologous to sequences of the invention. BLAST protein searches can be performed with the XBLAST program, score=50, wordlength=3, to obtain amino acid sequences homologous to protein sequences of the invention. To obtain gapped alignments for comparison purposes, Gapped BLAST can be utilized as described in Altschul et al., Nucleic Acids Res. 1997, 25:3389. Alternatively, PSI-Blast can be used to perform an iterated search that detects distant relationship between molecules. See Altschul et al. (1997) supra. When utilizing BLAST, Gapped BLAST, and PSI-Blast programs, the default parameters of the respective programs (e.g., XBLAST and NBLAST) can be used. See ncbi.nlm.nih.gov/BLAST/on the WorldWideWeb. Another non-limiting example of a mathematical algorithm utilized for the comparison of sequences is the algorithm of Myers and Miller, CABIOS 1988; 4: 11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

In a preferred embodiment, the percent identity between two amino acid sequences is determined using the algorithm of Needleman and Wunsch (J. Mol. Biol. 1970, 48:444-453), which has been incorporated into the GAP program in the GCG software package (Accelrys, Burlington, Mass.; available at accelrys.com on the WorldWideWeb), using either a Blossum 62 matrix or a PAM250 matrix, a gap weight of 16, 14, 12, 10, 8, 6, or 4, and a length weight of 1, 2, 3, 4, 5, or 6. In yet another preferred embodiment, the percent identity between two nucleotide sequences is determined using the GAP program in the GCG software package using an NWSgapdna.CMP matrix, a gap weight of 40, 50, 60, 70, or 80, and a length weight of 1, 2, 3, 4, 5, or 6. A particularly preferred set of parameters (and the one that can be used if the practitioner is uncertain about what parameters should be applied to determine if a molecule is a sequence identity or homology limitation of the invention) is using a Blossum 62 scoring matrix with a gap open penalty of 12, a gap extend penalty of 4, and a frameshift gap penalty of 5.

In addition to the cDNA sequences encoding various desired proteins, the present invention further provides polynucleotide molecules comprising nucleotide sequences having certain percentage sequence identities to any of the aforementioned sequences. Such sequences preferably hybridize under conditions of moderate or high stringency as described above, and may include species orthologs.

### SUBSTANTIALLY SIMILAR

The term “substantially similar” means a variant amino acid sequence preferably that is at least 80% identical to a native amino acid sequence, most preferably at least 90% identical.

### VARIANT

The term “variant” may also be used to indicate a modified or altered gene, DNA sequence, enzyme, cell, etc., i.e., any kind of mutant.

### PHARMACEUTICALLY ACCEPTABLE

When formulated in a pharmaceutical composition or formulation, a therapeutic compound of the present invention can be admixed with a pharmaceutically acceptable carrier or excipient. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human.

### PHARMACEUTICALLY ACCEPTABLE DERIVATIVE

The term “pharmaceutically acceptable derivative” as used herein means any pharmaceutically acceptable salt, solvate or prodrug, e.g. ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5th Edition, Vol 1: Principles and Practice, which is incorporated herein by reference to the extent of teaching such derivatives. Preferred pharmaceutically acceptable derivatives are salts, solvates, esters, carbamates, and phosphate esters. Particularly preferred pharmaceutically acceptable derivatives are salts, solvates, and esters. Most preferred pharmaceutically acceptable derivatives are salts and esters.

### PHARMACEUTICAL COMPOSITIONS, FORMULATIONS AND ADMINISTRATION

While it is possible to use a composition provided by the present invention for therapy as is, it may be preferable to administer it in a pharmaceutical formulation, e.g., in admixture with a suitable pharmaceutical excipient, diluent, or carrier selected with regard to the intended route of administration and standard pharmaceutical practice. Accordingly, in one aspect, the present invention provides a pharmaceutical composition or formulation comprising at least one active composition, or a pharmaceutically acceptable derivative thereof, in association with a pharmaceutically acceptable excipient, diluent, and/or carrier. The excipient, diluent and/or carrier must be “acceptable” in the sense of being compatible with the other ingredients of the formulation and not deleterious to the recipient thereof.

The compositions of the invention can be formulated for administration in any convenient way for use in human or veterinary medicine. In one embodiment, the MDSCs derived according to the methods of the present invention (i.e., active ingredient) can be delivered in a vesicle, including as a liposome (see Langer, Science, 1990; 249:1527-1533; Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.).

The effective amounts of compounds of the present invention include doses that partially or completely achieve the desired therapeutic, prophylactic, and/or biological effect. The actual amount effective for a particular application depends on the condition being treated and the route of administration. The effective amount for use in humans can be determined from animal models. For example, a dose for humans can be formulated to achieve circulating and/or gastrointestinal concentrations that have been found to be effective in animals.

### KITS

In one embodiment, the invention relates to a kit comprising ES-MDSC cells, optionally packaged in a manner suitable for clinical studies or for laboratory studies. In certain embodiments, the kits also include instructions teaching one or more of the methods described herein.

The abbreviations in the specification correspond to units of measure, techniques, properties or compounds as follows: “min” means minutes, “h” means hour(s), “μL” means microliter(s), “mL” means milliliter(s), “mM” means millimolar, “M” means molar, “μl” means microliter(s); “mmole” means millimole(s), “kb” means kilobase, “bp” means base pair(s), a.a. means “amino acid(s)”, and “IU” means International Units. “Polymerase chain reaction” is abbreviated PCR; “Reverse transcriptase polymerase chain reaction” is abbreviated RT-PCR; quantitative reverse transcriptase polymerase chain reaction is abbreviated “qPCR”, “Sodium dodecyl sulfate” is abbreviated SDS.

The proteins described herein are known by several names. The table below (Table 1) outlines these.

TABLE 1

| Name | Synonyms |
|---|---|
| Gr-1 | Lymphocyte antigen Ly-6G.1 and Ly-6C precursor (Ly6g) |
| F4/80 | Cell surface glycoprotein EMR1, Cell surface glycoprotein |
| | F4/80, DD7A5-7, EGF-TM7, EMR1 hormone receptor, |
| | F4/80, Gpf480, Ly71, TM7LN3 |
| CD11b | CD11b/CD18, Cell surface glycoprotein MAC-1 alpha |
| | subunit, CR3 alpha chain, F730045J24Rik, integrin alpha M, |
| | Leukocyte adhesion receptor MO1, Ly-40, Mac-1, Mac-1a |
| CD115 | Macrophage colony stimulating factor I receptor precursor |
| | (CSF1R), colony stimulating factor 1 receptor (c-fmsr), |
| | Fim-2, Fms, Fms proto-oncogene |
| SCF | Kitl, Kit ligand precursor, C-kit ligand, Clo, Con, contrasted, |
| | Gb, Hematopoietic growth factor KL, Kitlg, kit ligand, Mast |
| | cell growth factor, MGF, SCF, SF, Sl, SLF, Steel, Steel factor, |
| | Stem cell factor |
| BAFF | B-cell activating factor, also known as BLyS, TALL-1, |
| | THANK, zTNF4, TNFSF13B |
| M-CSF | SF1, MCSF, MGC31930, colony stimulating factor 1 |
| | (macrophage) |

“Myeloid-derived suppressor cells (MDSCs)”, also known as myeloid suppressor cells (MSCs), are a heterogeneous population of immature myeloid cells with immunoregulatory activity. The current term “MDSC” is used throughout the application and is synonymous with the term “MSC”. Human MDSCs are characterized by at least the expression of the cell markers CD11b and CD33. Human MDSCs may also express the markers CD15 and/or CD16. Murine MDSCs at least express the markers CD11b and Gr-1. Murine MDSCs may also express CD115 and/or F4/80 (see Li et al., Cancer Res. 2004, 64:1130-1139). Murine MDSCs may also express CD31, c-kit, vascular endothelial growth factor (VEGF)-receptor, or CD40 (Bronte et al., Blood. 2000, 96:3838-3846). MDSCs may further differentiate into several cell types, including macrophages, neutrophils, dendritic cells, Langerhand cells, monocytes or granulocytes. MDSCs may be found naturally in normal adult bone marrow of human and animals or in sites of normal hematopoiesis, such as the spleen in newborn mice.

Upon distress due to graft-versus-host disease (GVHD), cyclophosphamide injection, or γ-irradiation, for example, MDSCs may be found in the adult spleen. MDSCs can suppress the immunological response of T cells, induce T regulatory cells, and produce T cell tolerance. Morphologically, MDSCs usually have large nuclei and a high nucleus-to-cytoplasm ratio. MDSCs can secrete TFG-β and IL-10 and produce nitric oxide (NO) in the presence of IFN-γ or activated T cells. MDSCs may form dendriform cells; however, MDSCs are distinct from dendritic cells (DCs) in that DCs are smaller and express CD11c; MDSCs do not express CD11c, or express only a low level of CD11c. MDSCs can be isolated as described, e.g., in the co-pending application Ser. No. 12/159,929. T cell inactivation by MDSCs in vitro can be mediated through several mechanisms: IFN-γ-dependent nitric oxide production (Kusmartsevet al. J Immunol. 2000, 165: 779-785); Th2-mediated-IL-4/IL-13-dependent arginase 1 synthesis (Bronte et al. J Immunol. 2003, 170: 270-278); loss of CDξ signaling in T cells (Rodriguez et al. J Immunol. 2003, 171: 1232-1239); and suppression of the T cell response through reactive oxygen species (Bronte et al. J Immunol. 2003, 170: 270-278; Bronte et al. Trends Immunol. 2003, 24: 302-306; Kusmartsev et al. J Immunol. 2004, 172: 989-999; Schmielau and Finn, Cancer Res. 2001, 61: 4756-4760).

The term “embryonic stem (ES) cell” refers to a pluripotent stem cell derived from the inner cell mass of the blastocyst, an early-stage embryo. Embryonic stem cells are distinguished by two distinctive properties: their pluripotency and their capability to self-renew themselves indefinitely. ES cells are pluripotent, that is, they are able of differentiating into all derivatives of the three primary germ layers: ectoderm, endoderm, and mesoderm. Pluripotency distinguishes embryonic stem cells from adult stem cells found in adults; while embryonic stem cells can generate all cell types in the body, adult stem cells are multipotent and can only produce a limited number of cell types. Additionally, under defined conditions, embryonic stem cells are capable of propagating themselves indefinitely.

The term “hematopoietic stem cell (HSC)” refers to a cell that can give rise to all blood and lymphoid cell types including, for example, red blood cells, platelets, white blood cells, MDSCs, B cells, and T cells. HSCs can also propagate themselves, i.e., give rise to other HSCs, and may give rise to non-hematological cell types. HSC also have a long term reconstitution ability. HSCs are large cells that express Sca-1 and c-kit, have a high nucleus-to-cytoplasm ratio, and may express CD34.

Immune systems are classified into two general systems, the “innate” or “primary” immune system and the “acquired/adaptive” or “secondary” immune system. It is thought that the innate immune system initially keeps the infection under control, allowing time for the adaptive immune system to develop an appropriate response. Recent studies have suggested that the various components of the innate immune system trigger and augment the components of the adaptive immune system, including antigen-specific B and T lymphocytes (Kos, Immunol. Res. 1998, 17:303; Romagnani, Immunol. Today. 1992, 13: 379; Banchereau and Steinman, Nature. 1988, 392:245).

A “primary immune response” refers to an innate immune response that is not affected by prior contact with the antigen. The main protective mechanisms of primary immunity are the skin (protects against attachment of potential environmental invaders), mucous (traps bacteria and other foreign material), gastric acid (destroys swallowed invaders), antimicrobial substances such as interferon (IFN) (inhibits viral replication) and complement proteins (promotes bacterial destruction), fever (intensifies action of interferons, inhibits microbial growth, and enhances tissue repair), natural killer (NK) cells (destroy microbes and certain tumor cells, and attack certain virus infected cells), and the inflammatory response (mobilizes leukocytes such as macrophages and dendritic cells to phagocytose invaders).

Some cells of the innate immune system, including macrophages and dendritic cells (DC), function as part of the adaptive immune system as well by taking up foreign antigens through pattern recognition receptors, combining peptide fragments of these antigens with major histocompatibility complex (MHC) class I and class II molecules, and stimulating naive CD8⁺ and CD4⁺ T cells respectively (Banchereau and Steinman, supra; Holmskov et al., Immunol. Today. 1994, 15:67; Ulevitch and Tobias Annu. Rev. Immunol. 1995, 13:437). Professional antigen-presenting cells (APCs) communicate with these T cells, leading to the differentiation of naive CD4+ T cells into T-helper 1 (Th1) or T-helper 2 (Th2) lymphocytes that mediate cellular and humoral immunity, respectively (Trinchieri Annu. Rev. Immunol. 1995, 13:251; Howard and O'Garra, Immunol. Today. 1992, 13:198; Abbas et al., Nature. 1996, 383:787; Okamura et al., Adv. Immunol. 1998, 70:281; Mosmann and Sad, Immunol. Today. 1996, 17:138; O'Garra Immunity. 1998, 8:275).

A “secondary immune response” or “adaptive immune response” may be active or passive, and may be humoral (antibody based) or cellular that is established during the life of an animal, is specific for an inducing antigen, and is marked by an enhanced immune response on repeated encounters with said antigen. A key feature of the T lymphocytes of the adaptive immune system is their ability to detect minute concentrations of pathogen-derived peptides presented by MHC molecules on the cell surface.

In adaptive immunity, adaptive T and B cell immune responses work together with innate immune responses. The basis of the adaptive immune response is that of clonal recognition and response. An antigen selects the clones of cell which recognize it, and the first element of a specific immune response must be rapid proliferation of the specific lymphocytes. This is followed by further differentiation of the responding cells as the effector phase of the immune response develops. In T-cell mediated non-infective inflammatory diseases and conditions, immunosuppressive drugs inhibit T-cell proliferation and block their differentiation and effector functions.

The phrase “T cell response” means an immunological response involving T cells. The T cells that are “activated” divide to produce memory T cells or cytotoxic T cells. The cytotoxic T cells bind to and destroy cells recognized as containing the antigen. The memory T cells are activated by the antigen and thus provide a response to an antigen already encountered. This overall response to the antigen is the T cell response.

An “autoimmune disease” or “autoimmune response” is a response in which the immune system of an individual initiates and may propagate a primary and/or secondary response against its own tissues or cells. An “alloimmune response” is one in which the immune system of an individual initiates and may propagate a primary and/or secondary response against the tissues, cells, or molecules of another, as, for example, in a transplant or transfusion.

The term “cell-mediated immunity” refers to (1) the recognition and/or killing of virus and virus-infected cells by leukocytes and (2) the production of different soluble factors (cytokines) by these cells when stimulated by virus or virus-infected cells. Cytotoxic T lymphocytes (CTLs), natural killer (NK) cells and antiviral macrophages are leukocytes that can recognize and kill virus-infected cells. Helper T cells can recognize virus-infected cells and produce a number of important cytokines. Cytokines produced by monocytes (monokines), T cells, and NK cells (lymphokines) play important roles in regulating immune functions and developing antiviral immune functions.

A host T cell response can be directed against cells of the host, as in autoimmune disease. For example, the T cells in type I diabetes (T1D) recognize an “antigen” that is expressed by the host, which causes the destruction of normal host cells—for T1D, the endocrine 13-cells of the islets of Langerhans of the pancreas. A T cell response may also occur within a host that has received a graft of foreign cells, as is the case in graft-versus-host disease (GVHD) in which T cells from the graft attack the cells of the host, or in the case of graft rejection in which T cells of the host attack the graft.

A “T regulatory cell” or “Treg cell” or “Tr cell” refers to a cell that can inhibit a T cell response. Treg cells express the transcription factor Foxp3, which is not upregulated upon T cell activation and discriminates Tregs from activated effector cells. Tregs are identified by the cell surface markers CD25, CD45RB, CTLA4, and GITR. Treg development is induced by MDSC activity. Several Treg subsets have been identified that have the ability to inhibit autoimmune and chronic inflammatory responses and to maintain immune tolerance in tumor-bearing hosts. These subsets include interleukin 10-(IL-10-) secreting T regulatory type 1 (Tr1) cells, transforming growth factor-β-(TGF-β-) secreting T helper type 3 (Th3) cells, and “natural” CD4⁺/CD25⁺ Tregs (Trn) (Fehervari and Sakaguchi. J. Clin. Invest. 2004, 114:1209-1217; Chen et al. Science. 1994, 265: 1237-1240; Groux et al. Nature. 1997, 389: 737-742).

The phrase “inducing T regulatory cells” means activation, amplification, and generation of Tregs to inhibit or reduce the T cell response. One method of induction is through the use of the MDSCs.

The phrase “T cell tolerance” refers to the anergy (non-responsiveness) of T cells when presented with an antigen. T cell tolerance prevents a T cell response even in the presence of an antigen that existing memory T cells recognize.

The term “differentiate” refers to the genetic process by which cells are produced with a specialized phenotype. A differentiated cell of any type has attained all of the characteristics that define that cell type. This is true even in the progression of cell types. For example, if cell type X matures to cell type Y which then overall matures to cell type Z, an X cell differentiates to a Y cell when it has attained all of the characteristics that define a type Y cell, even though the cell has not completely differentiated into a type Z cell.

The term “SHIP” refers to (SRC-homology-2-domain-containing inositol-5-phosphatase). SHIP catalyzes the hydrolysis of the membrane inositol lipid PIP3, thereby preventing activation of PLCγ and Tec kinases and abrogating the sustained calcium flux mediated by the influx of calcium through the capacitance coupled channel. SHIP signaling is known to affect maturation of MSCs (Ghansah et al. J. Immunol. 2004, 173:7324-7330).

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

“Cytokine” is a generic term for a group of proteins released by one cell population which act on another cell population as intercellular mediators. Examples of such cytokines are lymphokines, monokines, and traditional polypeptide hormones. Included among the cytokines are interferons (IFN, notably IFN-γ), interleukins (IL, notably IL-1, IL-2, IL-4, IL-10, IL-12), colony stimulating factors (CSF), macrophage colony stimulating factor (M-CSF), granulocyte macrophage colony stimulating factor (GM-CSF), thrombopoietin (TPO), erythropoietin (EPO), leukemia inhibitory factor (LIF), kit-ligand, growth hormones (GH), insulin-like growth factors (IGF), parathyroid hormone, thyroxine, insulin, relaxin, follicle stimulating hormone (FSH), thyroid stimulating hormone (TSH), leutinizing hormone (LH), hematopoietic growth factor, hepatic growth factor, fibroblast growth factors (FGF), prolactin, placental lactogen, tumor necrosis factors (TNF), mullerian-inhibiting substance, mouse gonadotropin-associated peptide, inhibin, activin, vascular endothelial growth factor (VEGF), integrin, nerve growth factors (NGF), platelet growth factor, transforming growth factors (TGF), osteoinductive factors, etc. Those of particular interest for the present invention include IFN-γ, IL-10, and TGF-β.

“Autoantigen” refers to a molecule that is endogenous to a cell or organism that induces an autoimmune response.

“Transplant rejection” means that a transplant of tissue or cells is not tolerated by a host individual. The transplant is not tolerated in that it is attacked by the host's own immune system or is otherwise not supported by the host. The transplant may be an allotransplant, a transplant of tissue or cells from another individual of the same species, or an autotransplant, a transplant of the host's own tissue or cells. Transplant rejection encompasses the rejection of fluids through transfusion.

The term “subject” or “individual” as used herein refers to an animal having an immune system, preferably a mammal (e.g., rodent such as mouse). In particular, the term refers to humans.

Administration

The compositions and formulations of the present invention can be administered topically, parenterally, orally, by inhalation, as a suppository, or by other methods known in the art. The term “parenteral” includes injection (for example, intravenous, intraperitoneal, epidural, intrathecal, intramuscular, intraluminal, intratracheal or subcutaneous). The preferred routes of administration is intravenous (i.v.).

In certain embodiments, the present invention provides methods of treating a disorder amenable to cell-based treatment in a mammal, comprising administering a pharmaceutically effective amount of an isolated MDSC of the invention to a mammal in need thereof.

Examples of disorders that are amenable to cell-based treatment include, but are not limited to autoimmune diseases and alloimmune responses. Non-limiting examples of disorders that may be treated using the methods of the present invention, include conditions in which the immune system of an individual (e.g., activated T cells) attacks the individual's own tissues and cells, or implanted tissues, cells, or molecules (as in a graft or transplant). Exemplary autoimmune diseases that can be treated with the methods of the instant disclosure include type I diabetes, multiple sclerosis, thyroiditis (such as Hashimoto's thyroiditis and Ord's thyroiditis), Grave's disease, systemic lupus erythematosus, scleroderma, psoriasis, arthritis, rheumatoid arthritis, alopecia greata, ankylosing spondylitis, autoimmune hemolytic anemia, autoimmune hepatitis, Behcet's disease, Crohn's disease, dermatomyositis, glomerulonephritis, Guillain-Barre syndrome, IBD, lupus nephritis, myasthenia gravis, myocarditis, pemphigus/pemphigoid, pernicious anemia, polyarteritis nodosa, polymyositis, primary biliary cirrhosis, rheumatic fever, sarcoidosis, Sjogren's syndrome, ulcerative colitis, uveitis, vitiligo, and Wegener's granulomatosis. Exemplary alloimmune responses that can be treated with the methods of the instant disclosure include GVHD and transplant rejection.

Administration of the compositions of the invention may be once a day, twice a day, or more often, but frequency may be decreased during a maintenance phase of the disease or disorder, e.g., once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. More generally, dose and frequency will depend in part on recession of pathological signs and clinical and subclinical symptoms of a disease condition or disorder contemplated for treatment with the present compounds.

It will be appreciated that the amount of MDSCs of the invention required for use in treatment will vary with the route of administration, the nature of the condition for which treatment is required, and the age, body weight and condition of the patient, and will be ultimately at the discretion of the attendant physician or veterinarian. These compositions will typically contain an effective amount of the compositions of the invention, alone or in combination with an effective amount of the MDSCs of the invention. Preliminary doses can be determined according to animal tests, and the scaling of dosages for human administration can be performed according to art-accepted practices.

Keeping the above description in mind, typical dosages of MDSCs of the invention for administration to humans range from about 5×10⁵ to about 5×10¹¹/kg or higher, although lower or higher numbers of MDSCs are also possible. In a preferred embodiment, a 100 kg patient may receive, for example, 5×10⁷-5×10¹³ MDSCs.

### Materials and Methods

The following describes the materials and methods employed in Examples 1-6.

ES Cell Maintenance

Generation of HoxB4-transduced ES cell line (HoxB4-ESs, 129SvEv background) has been described previously. (Kyba M, et al., (2002) “HoxB4 confers definitive lymphoid-myeloid engraftment potential on embryonic stem cell and yolk sac hematopoietic progenitors”; Cell; April 5; 109(1):29-37). The expansion and maintenance of HoxB4-ES cells were conducted according to methods well established. (Kennedy M, Keller G M. Hematopoietic commitment of ES cells in culture. Methods Enzymol. 2003; 365:39-59). Briefly, HoxB4-ESs were plated onto irradiated mouse embryonic fibroblast (MEF) cells and incubated in Dulbecco's Modified Eagle Medium (DMEM) supplemented with 15% fetal calf serum (ES-qualified, Gemcell), leukemia inhibitory factor (LIF, 0.5% conditioned medium, CM), and 1.5×10⁻⁴ M monothioglycerol (MTG). The ES/MEF co-cultures were fed every other day with fresh medium until for use.

Differentiation of ES Cells into Myeloid-Derived Suppressor Cells

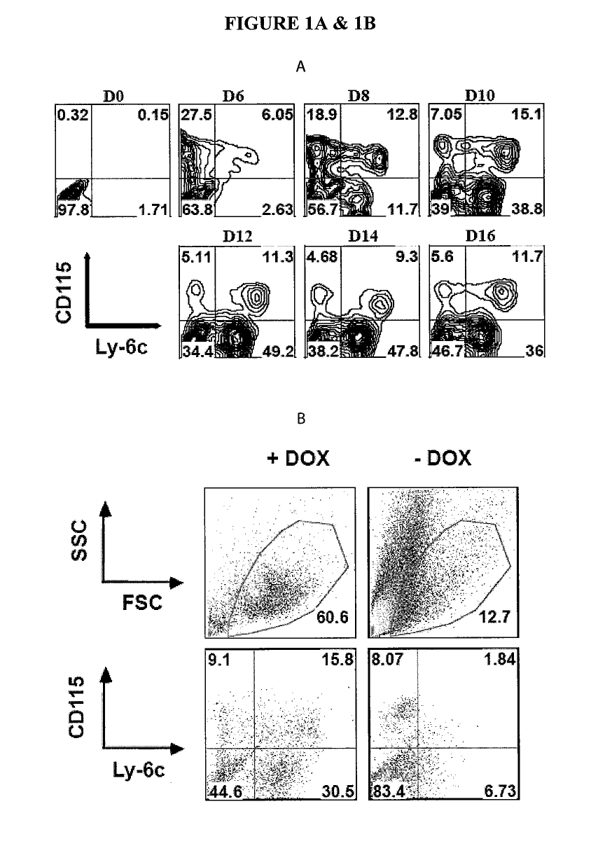

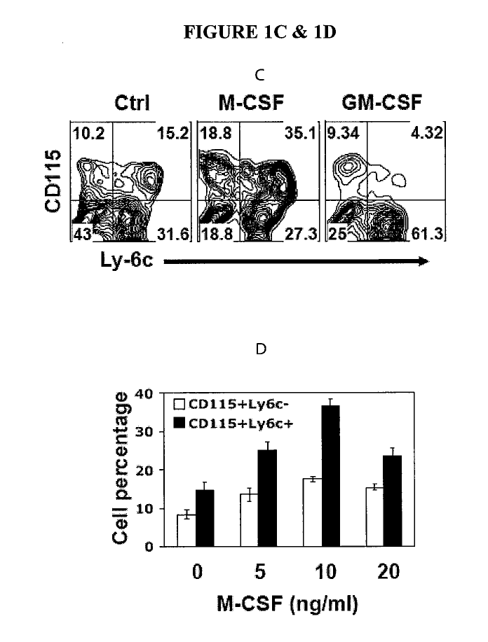

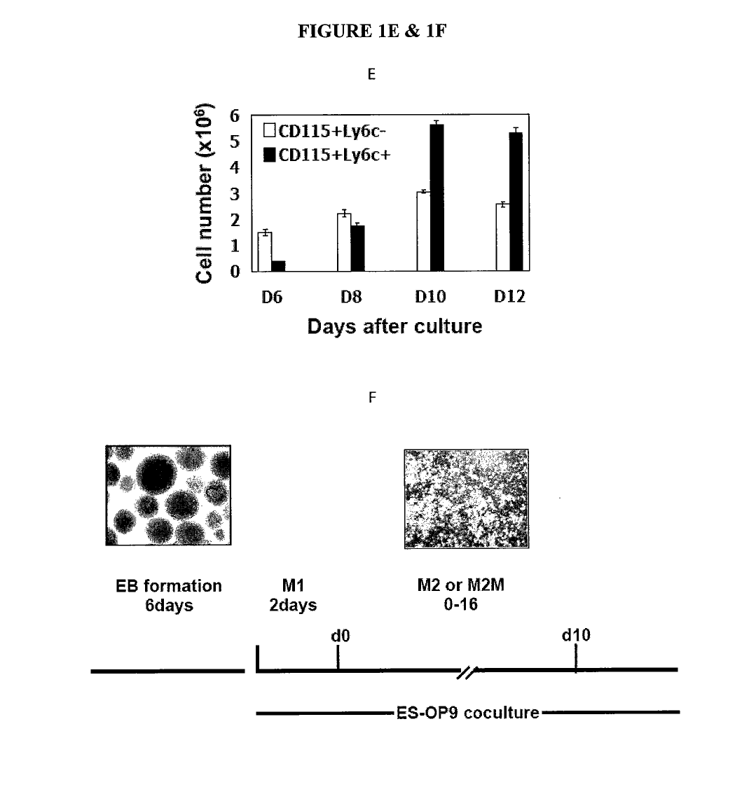

A 3-step differentiation strategy was designed to generate MDSCs from HoxB4-ESs as outlined in FIG. 1F. Firstly, HoxB4-ESs were induced to undergo differentiation through a well-characterized process named embryonic bodies (EBs) formation. Two days prior to this step, freshly expanded HoxB4-ESs were recovered from the ES/MEF cocultures and passaged twice on gelatinized plates. Colonies with proper density were then digested with trypsin, centrifuged, and resupended in Iscove's Modified Dulbecco's Medium (IMDM) supplemented with 15% FCS, 10% protein-free hybridoma medium (PFHM-II; GIBCO/BRL), 2 mM L-glutamine, transferrin (300 μg/ml), ascorbic acid (50 μg/ml), and 4.5×10⁻⁴ M MTG. The cells were plated at 3×10³ cells/ml into low-cluster dishes (Costar, 35 mm, 5 ml/dish) to form EBs without changing the medium throughout the culture time.

Next, EB-derived cells were subjected to further differentiation by co-culturing them with OP9 stroma, an M-CSF-deficient cell line that has been widely used for in vitro differentiation of ES cells. For this, day 6 EBs (except otherwise specified) were trypsinized, and dissociated cells were seeded onto semiconfluent OP9 monolayers (irradiated immediately before coculture) at a density of 1×10⁵/ml (2 ml/well, 6-well plate), and cultured in medium containing IMDM, 15% FCS (BenchMark, Gemcell), 2 mM L-glutamine, 100 U/mL penicillin and 100 μg/mL streptomycin, 1 ug/ml doxycycline, 1.5×10⁻⁴M MTG, plus M1 cytokine mix composed of 1% c-kit Ligand (KL; CM), 10 mg/ml murine IL-6, and 0.5% WEHI (IL-3, CM). 2 days later, the medium was removed and replaced with 3 ml of fresh medium containing M2 cytokine mix consisting of 1% KL supernant, 0.5% murine thrombopoietin (TPO, CM), 40 ng/ml murine VEGF, 50 ng/ml murine Flt-3L or with medium containing M2 plus 10 ng/ml murine M-CSF (or M2+M-CSF) (all mouse recombinant cytokines were purchased from Peprotec Inc.), and medium was changed every other day thereafter. Unless otherwise specified, non-adherent and loosely attached cells were harvested after 10 days coculture in M2+M-CSF and used for morphologic, phenotypic, developmental, and functional analysis.

Redifferentiation and Colony-Forming Assay

For redifferentiation, FACS-sorted CD115⁺Ly-6C⁻ and CD115⁺Ly-6C⁺ cells were plated onto gelatinized plate (96-well, 5×10⁴/well) and cultured in IMDM supplemented with 10% FCS (BenchMark, Gemcell), 2 mM L-glutamine, 100 U/mL penicillin and 100 μg/mL streptomycin, 1.5×10⁻⁴ M MTG, and with the following murine growth factors: 5 mg/ml IL-6, 0.5% IL-3 (CM), 10 ng/ml IL-β, 10 ng/ml bFGF, 10 ng/ml erythropoietin (EPO), 0.5% TPO (CM), 20 ng/ml VEGF, 25 ng/ml Flt-3L, 10 ng/ml M-CSF, 1% GM-CSF (CM). For the experiments examining the role of M-CSF and GM-CSF, one of the two cytokines or both was omitted. Cells were collected on the indicated days and analyzed by FACS for the expression of CD115 and Ly6c, or cytospun onto slides and stained with May-Grünwald Giemsa. To evaluate colony-forming activity of CD115⁺Ly-6C⁻ and CD115⁺Ly-6C cells, the same medium for redifferentiation was used and 1% sterilized methylcellulose was added to it. After mixed vigorously with FACS-sorted cells, the cell-media mixture was then dispensed in triplicates into 35-mm dishes (1×10⁴ cells/1.1 ml/dish) and incubated at 37° C. with a>95% humidity. Colonies were scored at day 9 under an inverted microscope and distinguished into following categories: colony-forming unit (CFU)-macrophage (M), CFU-granulocyte (G), and CFU-granulocyte, macrophage (GM). Pooled colonies from each dish were stained by May-Grünwald Giemsa for morphological evaluation.

Flow Cytometric Analysis and Cell Sorting

All fluorochrome-labeled anti-mouse antibodies were purchased from eBioscience (San Diego, Calif.) or BD Biosciences (San Diego, Calif.). Surface staining was performed as described. Pan P Y, et al. (2008) “Reversion of immune tolerance in advanced malignancy: modulation of myeloid-derived suppressor cell development by blockade of stem-cell factor function,” Blood; 111:219-228. Intracellular staining of Foxp3 was conducted per the manufacturer's instructions (Mouse Regulatory T cell Staining Kit, eBioscience). Data were acquired on a FACSAria™ or FACS LSR-II (BD Biosciences) and analyzed using Flowjo software (Tree Star, Inc., Ashland, Oreg.). To purify total CD115⁺ cells from ES or HS cell cultures, samples were stained with anti-CD115 antibodies, and CD115 positive cells were isolated using magnetic beads (Miltenyi Biotec, Auburn, Calif.). In some experiments, bulk CD115⁺ cells were further FACS-sorted into CD115+Ly-6C⁻ and CD115+Ly-6C⁺ cells. The purity of the sorted cells was checked by flow cytometry and sorted cells with a purity of greater than 90% (for bead-sorted cells) or 98% (for FACS-sorted cells) were used.

Suppression Assay and Treg Induction

Tumor-derived MDSCs (TD-MDSCs) were prepared as described (Pan P Y, et al. Reversion of immune tolerance in advanced malignancy: modulation of myeloid-derived suppressor cell development by blockade of stem-cell factor function. Blood. 2008; 111:219-228). Briefly, BM cells isolated from tumor-bearing 129SvEv mice (F9 mouse embryonal carcinoma) or C57BL/6 (B6) mice (Lewis lung carcinoma) were fractionated via Percoll and cell bands between 50% and 60% were collected for enrichment of CD115⁺ cells using magnetic beads (Miltenyi Biotec, Auburn, Calif.). Because CD115⁺ cells homogenously express Gr-1 and F4/80, CD115 positive-selected cells with a purity of greater than 90% were used as TD-MDSCs.

The suppressive activity of MDSCs was assessed either by co-culturing 1×10⁵ 129SvEv (using B6 for HSC-MDSCs comparison) splenocytes with various numbers of CD115⁺ cells or CD115⁺ cells for 3 days in the presence of anti-CD3/anti-CD28 (0.5 μg/ml) or by co-culturing 2×10⁵ 129SvEv splenocytes (responder) with an equal number of irradiated (25 Gy) BALB/c splenocytes (stimulator) in the presence of various numbers of CD115⁺ or CD115⁻ cells for 4 days. [³H]-thymidine was added for the last 8 hours of incubation. For Treg induction, 4×10⁶/well of 129SvEv (B6 for HSC-MDSCs) splenocytes and 1×10⁶ CD115⁺ or CD115⁻ cells were cocultured for 5 days in the presence of anti-CD3/anti-CD28 (0.5 μg/ml). Foxp3 expression was determined by intracellular staining and NO level in the supernatant was measured by Greiss reagents (Sigma-Aldrich, St. Louis, Mo.).

Reverse Transcription-Polymerase Chain Reaction.

Magnetic bead-purified cells were left untreated (ctrl) or stimulated with IFN-γ (50 ng/ml) or IL-13 (40 ng/ml). 24 h later, cell pellets were lysed with TRIzol reagent for RNA isolation. A one-step RT-PCR kit (Qiagen) was used for reverse transcription of RNA and amplification of cDNA (30 cycles for all analysis). The primers for genes examined were indicated as following: (SEQ ID NO:5) iNOS 5′-GAGATTGGAGTTCGAGACTTCTGTG-3′ (sense) and (SEQ ID NO:6) 5′-TGGCTAGTGCTTCAGACTTC-3′ (antisense); (SEQ ID NO:7) arginase 1 5′-CAGAGTATGACGTGAGAGACCAC-3′ (sense) and (SEQ ID NO:8) 5′-CAGCTTGTCTACTTCAGTCATGGAG-3′ (antisense); (SEQ ID NO:9) IL-10 5′-CTCTTACTGACTGGCATGAGG-3′ (sense) and (SEQ ID NO:10) 5′-CCTTGTAGACACCTTGGTCTTGGAG-3′ (antisense); (SEQ ID NO:11) TGF-β1 5′-GTGGTATACTGAGACACCTTGG-3′(sense) and (SEQ ID NO:12) 5′-CCTTAGTTTGGACAGGATCTGG-3′ (antisense); and (SEQ ID NO:13) GAPDH 5′-GTGGAGATTGTTGCCATCAACG-3′ (sense) and (SEQ ID NO:14) 5′-CAGTGGATGCAGGGATGATGTTCTG-3′ (antisense).

Allogeneic BM Transplantation

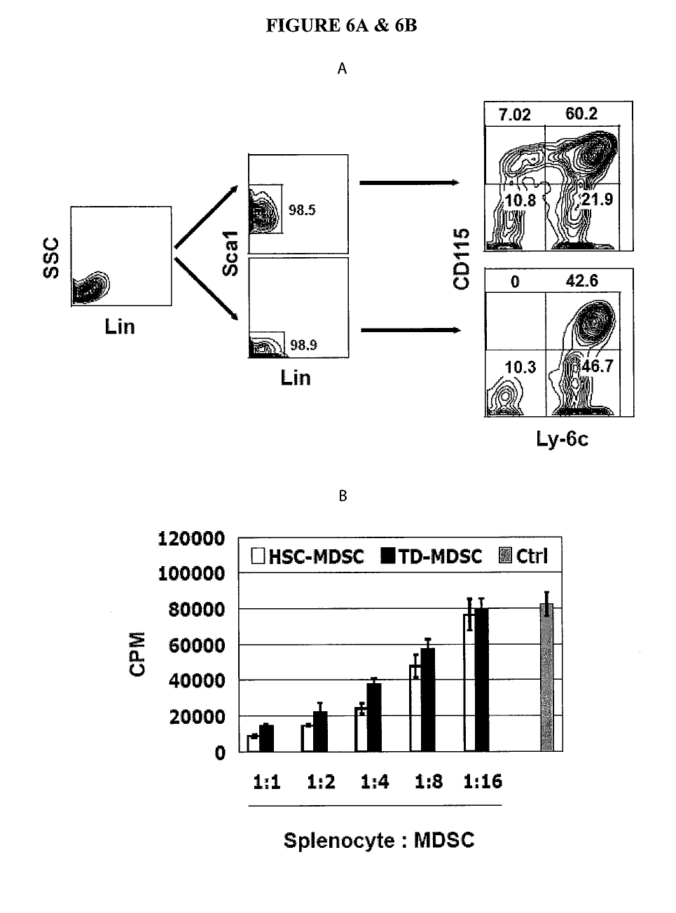

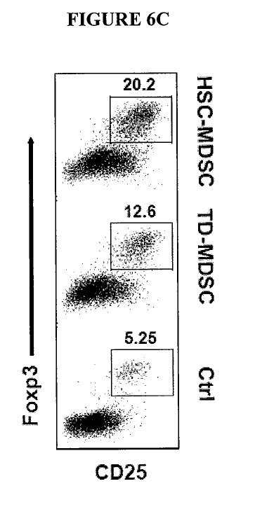

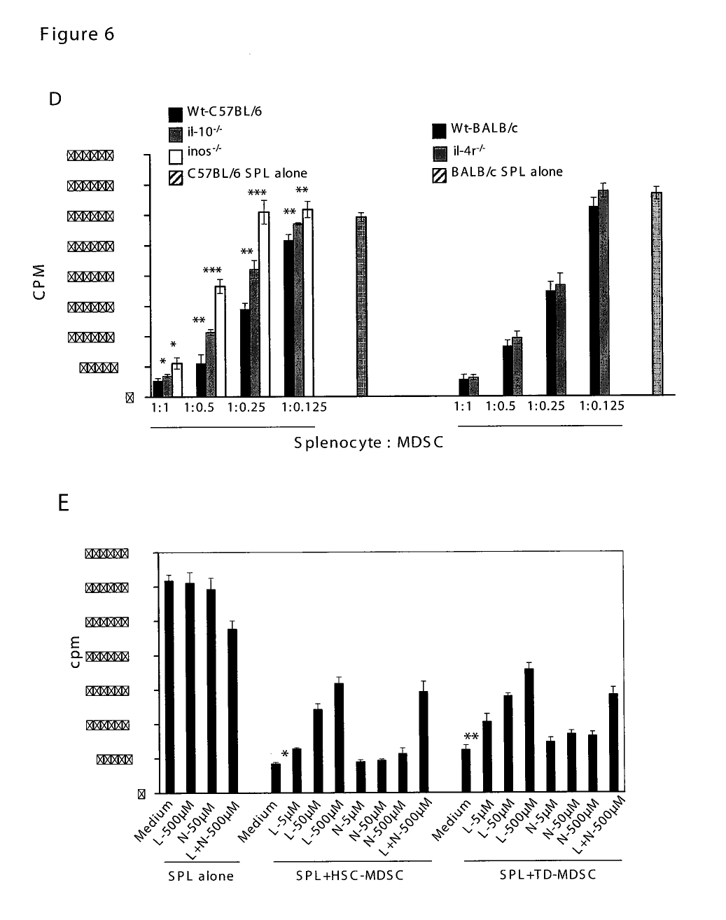

To prepare T cell-depleted bone marrow cells (TCDBM), BM cells isolated from naïve 129SvEv mice were purified using anti-Thy-1.2 antibodies, and Thy-1.2 negative cells were used as TCDBM. Donor T cells (T) were prepared from splenocytes of naïve 129SvEv mice using a negative selection kit (R&D systems, Minneapolis, Minn.). For establishment of the GvHD model, BALB/c mice (8-10 weeks old) were lethally irradiated (¹³⁷Cs source, 8Gy, TBI, split in twice treatments with an 4-h interval). Within 24 hours after irradiation, recipients were left nongrafted or reconstituted via tail vein injection with donor-derived cells as detailed in FIG. 6A. ES-MDSC-treated mice were given two additional infusions of ES-MDSCs (bulk CD115⁺ cells, 2×10⁶/mouse, each) on d4 and d10, respectively. Animals were monitored daily for GVHD induction and overall survival or were weighed every other 3˜4 days. For histopathological analysis, specimens obtained on d23 were fixed in formalin and tissue sections were stained with hematoxylin and eosin.

Derivation of MDSCs from Marrow Hematopoietic Stem/Progenitor Cells

BM cells prepared from naïve B6 mice were depleted of lineage positive cells using antibodies against a panel of lineage antigens including CD5, CD45R CD11b, Gr-1 (Ly-6G/C), and Ter-119 (Miltenyi Biotec, Auburn, Calif.). The Lin⁻ cells were further fractioned into Sca1⁺ and Sca1⁻ populations by FACS sorting. To derive MDSCs, similar culture conditions were used as indicated in generation of MDSCs from ES cells. The highly purified Sca1⁺ and Sca1⁻ cells were separately plated at a density of 2.5×10⁵/ml (24-well plate) and cultured in M1 (see above) for 2-4 days, depending on the cell number required for subsequent differentiation. The expanded stem/progenitor cells were then transferred to 6 well plates (3×10⁵/2 ml/well) and incubated in M2+M-CSF (see above) for various days.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Myeloid-Derived Suppressor Cells can be Generated Efficiently from ES Cells

The HoxB4 ES cell line (Kyba et al.)) was used as starting cells for generation of MDSCs. Transduction with HoxB4, a member of the Hox family of homeodomain transcription factors, has been shown to bias ES cells differentiation to myeloid development over a wide range of ectopic expression levels. (Pilat S, Carotta S, Schiedlmeier B, et al. HOXB4 enforces equivalent fates of ES-cell-derived and adult hematopoietic cells. Proc Natl Acad Sci USA. 2005; 102:12101-12106.) The CD115⁺Gr1⁺F4/80⁺ cells were also identified as a major component of MDSCs in tumor-bearing mice. Thus, the present experiments were conducted to establish a differentiation condition conducive to the induction of the CD115⁺Gr1⁺F4/80⁺ population from ES cells. A three-stage differentiation strategy was utilized, as described in the Methods section. The cytokine requirement of MDSC development was targeted for particular evaluation. After a 7-day (total 9 days) co-culture of day 6 EB-disaggregated cells with OP9 stromal cell lines, a considerable portion of cells co-expressing CD115 and Ly-6C was detected from cultures with presence of M2 cocktail (KL, VEGF, Flt3L, and TPO), but absent from cultures with a M3 cytokine mix consisting of KL, IL-6, IGF, IL-β.

Kinetic analysis indicated that the CD115+Ly-6C⁺ population reached peak frequency on day 10 after co-culture with OP9 stroma in M2 and remained at relatively higher levels thereafter (FIG. 1A). Hoxb4 ES cells were first grown in suspension to form embryonic bodies (EB), and cells dissociated from D6 EB were plated onto semiconfluent (irradiated) OP9 stromal cells and cultured in medium 1 (M1, see details in Methods). After 48 hours, M1 medium was removed and replaced with medium 2 (M2, see details in Methods). Floating and loosely attached cells recovered on different days after co-culture in M2 were stained and analyzed by FACS.

Interestingly, an additional CD115 positive but Ly6c negative population was also observed with a different kinetics of emergences under these culture conditions (FIG. 1A). EBs were also compared at different stages (day-5,6,7,9) and the results illustrated that day 6 EBs resulted in highest percentage of CD115⁺ cells, especially for the population of CD115⁺Ly-6C⁺ cells. To assess a potential role of HoxB4, ES cells were cultured by taking advantage of the tetracycline-inducible expression of hoxb4, in the presence or absence of doxycycline (DOX). As determined by FSC vs SSC, cells incubated without induction of hoxb4 expression did not grow well, resulting in nearly 90 percent dead cells (FIG. 1B, upper panel). Among the residual surviving cells, while the development of CD115⁺Ly-6C⁻ cells was not significantly affected, CD115⁺Ly-6C⁺ cells and CD115⁻Ly-6C⁺ cells were severely reduced in the absence of DOX (FIG. 1B, lower panel), suggesting that HoxB4 is required for both survival and differentiation of myeloid lineages derived from the ES cell line.

To enhance the production of ES cell-derived myeloid cells, additional cytokines were tested, including the colony-stimulating factor family members M-CSF and GM-CSF. As shown in FIG. 1C, addition of GM-CSF to the cultures led to a dramatic decrease of CD115+Ly-6C⁺ cells and a marked increase of CD115⁻Ly-6C⁺ cells, respectively (FIG. 1C, right plot), suggesting that GM-CSF might accelerate the transition of CD115+Ly-6C⁺ cells into their progeny or skewed the CD115+Ly-6C⁻ cells into granulocytes. For experiments in FIGS. 1C and D, Hoxb4 ES cells were differentiated with the same procedure as described in FIG. 1A, but in the last step cells were cultured for 10 day in M2 or M2 supplanted with M-CSF or GM-CSF (FIG. 1C) or M2 plus various concentration of M-CSF (FIG. 1D).

In contrast, the production of both CD115⁺ populations was significantly augmented in the presence of M-CSF (FIG. 1C, middle plot), and the effect of M-CSF was manifested in dose-dependent manner (FIG. 1D). However, the percentages of CD115⁺ cells were decreased at higher concentration of M-CSF (20 ng/ml), likely resulting from the competitive binding to the CD115 (M-CSF receptor) between M-CSF and anti-CD115 antibodies used for flow cytometric staining. In view of the favorable effects of M-CSF, the M2 cytokine cocktail plus 10 ng/ml of M-CSF (designated as M2M) was used in the subsequent experiments for deriving MDSCs. With this modified culture condition, roughly 5.6×10⁶ CD115+Ly-6C⁺ and 2.9×10⁶ CD115+Ly-6C⁺ cells could be acquired from 3×10⁵ EB cells (1×10⁵/well, 6-well plate) after a 10-day differentiation in M2M (M2+M-CSF) (FIG. 1E). At the indicated dates in M2 plus 10 ng/ml of M-CSF (M2M), floating and loosely adherent cells were retrieved and cell numbers were determined based on cell count and percentage of individual population. Error bars represent standard deviations (n=3).

### Example 2

### ES Cell-Derived CD115⁺ Cells Exhibit Strong Suppressive Capacity In Vitro

Immune suppression is the hallmark feature of MDSCs. Using HoxB4 ES cell line, MDSCs expressing both CD115⁺ and Ly-6C⁺ have been generated as described in Example 1. These ES cell-derived myeloid cells were tested for their functional characteristics. Differentiated cells were separated into CD115⁺ and CD115⁻ cells using magnetic beads and co-cultured with splenocytes isolated from naïve 129SvEv mice.

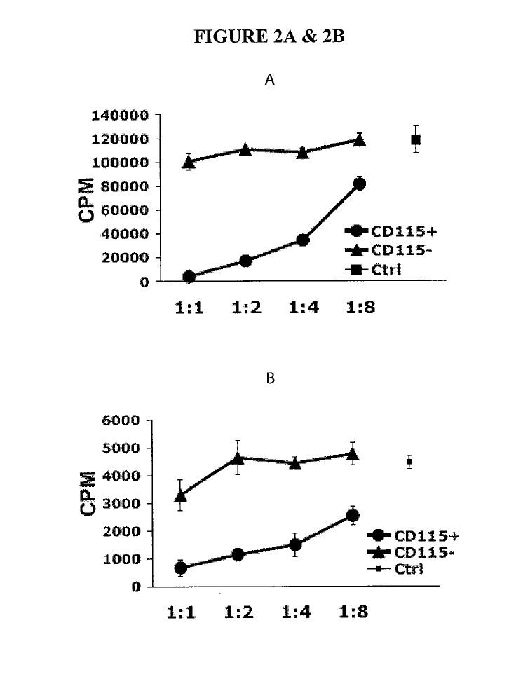

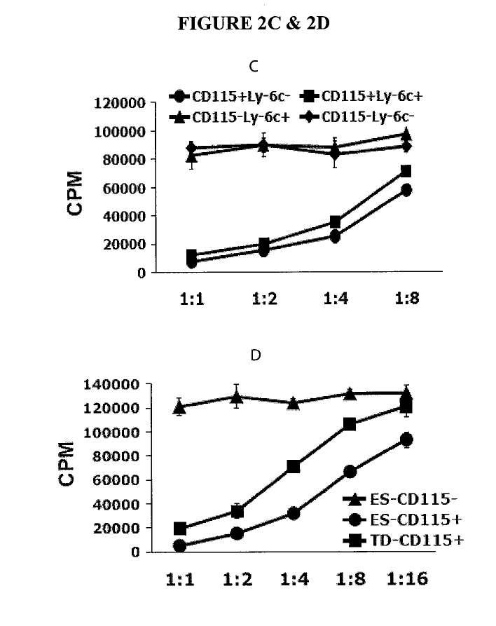

Remarkably, HoxB4 ES cell-derived CD115⁺ but not CD115⁻ cells displayed potent suppressive activity against T cell proliferation stimulated either by stimulus with anti-CD3 plus anti-CD28 (FIG. 2A) or by allo-antigens in a mixed lymphocyte reaction (MLR) setting (FIG. 2B). Because the CD115⁺ fraction was composed of two populations, CD115⁺Ly-6C⁺ and CD115⁺Ly-6C⁻ cells, the two subsets were purified by FACS sorting and conducted similar proliferation assays. Various numbers of ES cell-derived cells isolated or TD-CD115⁺ cells were co-incubated with 129SvEv splenocytes in the presence of anti-CD3/anti-CD28 (FIG. 2 A, C, D) or irradiated BALB/c splenocytes (FIG. 2B) and [³H]-thymidine was pulsed for the final 8 hours of a 3-day (FIG. 2 A, C, D) or a 4-day (FIG. 2B) culture. Data shown in FIGS. 2A-D are representative of at least three experiments with consistent results.

Sorted CD115⁻Ly-6C⁻ and CD115⁻Ly-6C⁺ populations showed minimal suppressor function. However, both CD115⁺Ly-6C⁻ and CD115⁺Ly-6C⁺ populations exerted robust inhibition of T cell proliferation with the former showing even slightly higher suppressive activity (FIG. 2C). The suppressive capacity of ES cell-derived CD115⁺ cells (ES-CD115⁺) versus those isolated from tumor-bearing mice (TD-CD115⁻) was further compared. Surprisingly, ES-CD115⁺ cells were even more suppressive than the TD-CD115⁺ cells (FIG. 2D). Thus, both CD115⁺Ly-6C⁺ and CD115⁺Ly-6C⁻ subsets exhibited more suppressive activity than the TD-CD115⁺ cells.

These results clearly indicate that two functionally active MDSC populations can be generated from ES cells in vitro and these are designated as ES-MDSCs.

### Example 3

### The Suppressor Function of ES-MDSCs Involves Multiple Pathways

MDSCs are known to suppress T-cell responses directly via diverse mechanisms, e.g. production of nitric oxide (NO), expression of arginase and nitric oxide synthetase (NOS), two inducible enzymes regulating arginine metabolism, and/or secretion of IL-10 and TGF-β (Sinha P, Clements V K, Bunt S K, Albelda S M, Ostrand-Rosenberg S. Cross-talk between myeloid-derived suppressor cells and macrophages subverts tumor immunity toward a type 2 response. J Immunol. 2007; 179:977-983, Bronte V, Zanovello P. Regulation of immune responses by L-arginine metabolism. Nat Rev Immunol. 2005; 5:641-654, Rodriguez P C, Ochoa A C. Arginine regulation by myeloid derived suppressor cells and tolerance in cancer: mechanisms and therapeutic perspectives. Immunol Rev. 2008; 222:180-191, Terabe M, Matsui S, Park J M, et al. Transforming growth factor-beta production and myeloid cells are an effector mechanism through which CD1d-restricted T cells block cytotoxic T lymphocyte-mediated tumor immunosurveillance: abrogation prevents tumor recurrence. J Exp Med. 2003; 198:1741-1752, and Nagaraj S, Gupta K, Pisarev V, et al. Altered recognition of antigen is a mechanism of CD8+ T cell tolerance in cancer. Nat Med. 2007; 13:828-835) or indirectly by inducing T regulatory cell development. (Huang B, Pan P Y, Li Q, et al. Gr-1+CD115+ immature myeloid suppressor cells mediate the development of tumor-induced T regulatory cells and T-cell anergy in tumor-bearing host. Cancer Res. 2006; 66:1123-1131, Serafini P, Mgebroff S, Noonan K, Borrello I. Myeloid-derived suppressor cells promote cross-tolerance in B-cell lymphoma by expanding regulatory T cells. Cancer Res. 2008; 68:5439-5449, and Ghiringhelli F, Puig P E, Roux S, et al. Tumor cells convert immature myeloid dendritic cells into TGF-beta-secreting cells inducing CD4+CD25+ regulatory T cell proliferation. J Exp Med. 2005; 202:919-929.)

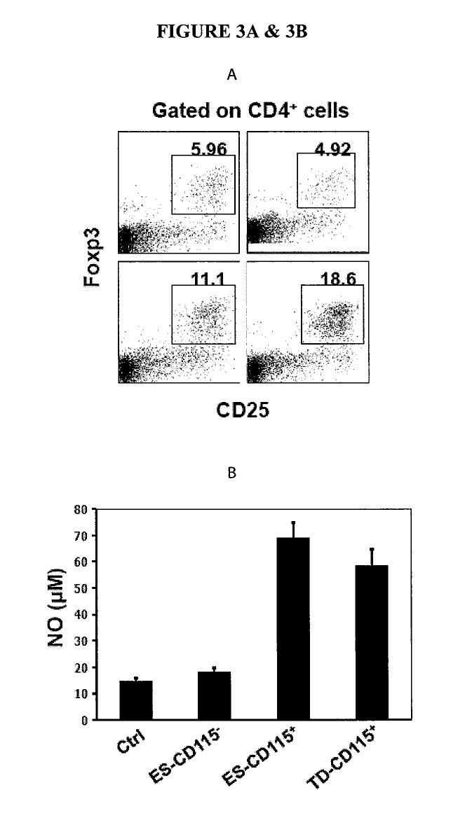

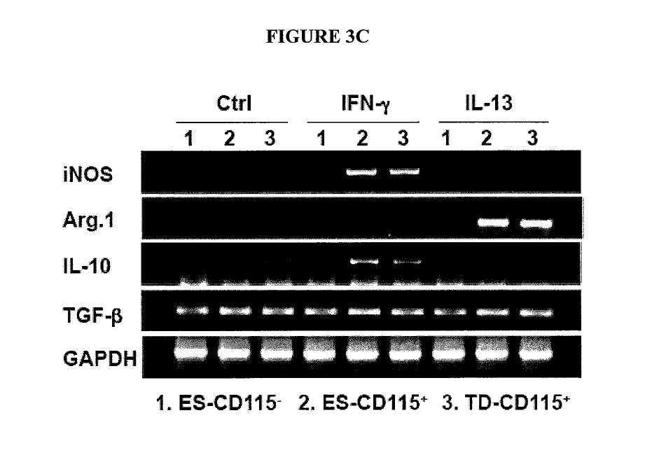

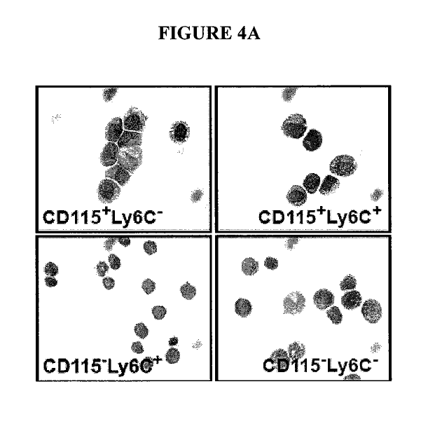

To delineate which of the aforementioned pathways is involved in mediating the T-cell suppression, the ES-MDSCs were analyzed for their ability to induce Tregs and production of NO. Interestingly, while both ES-CD115⁺ (FIG. 3A, lower right) and TD-CD115⁺ (FIG. 3A, lower left) cells induced drastic propagation of Tregs, the percentage of Tregs was significantly higher (P<0.001, student test) in the presence of ES-CD115⁺ cells compared to TD-CD115⁺ cells. Similarly, high amounts of NO were only detected in the supernatants collected from cultures of ES-CD115⁺ or TD-CD115⁺ cells (FIG. 3B). In FIGS. 3A and B, 1×10⁶ of ES cell-derived CD115⁺ and CD115⁻ cells or TD-CD115⁺ cells were co-incubated with 4×10⁶ 129SvEv splenocytes for 5 days in the presence of anti-CD3/anti-CD28. CD4⁺CD25⁺Foxp3⁺ cells were analyzed by intracellular staining. NO levels in the supernatants were determined per manufacture's protocol (n=3).

In contrast, addition of ES-CD115⁻ cells showed no apparent change in the frequency of Tregs (FIG. 3A, upper right) compared to splenocyte-only cultures (FIG. 3A, upper left).

The expression of a panel of genes including iNOS, Arginase 1, IL-10, and TGF-β was also assessed. ES cell-derived CD115⁺ and CD115⁻ cells and TD-CD115⁺ cells were cultured for 24 hrs in the presence or absence of IFN-γ or IL-13. mRNA expressions were analyzed by RT-PCR. As shown in FIG. 3C, both ES-CD115⁺ and TD-CD115⁺ cells constitutively expressed high levels of TGF-β and low levels of IL-10 that was considerably enhanced in the presence of IFN-γ. (Huang B, Pan P Y, Li Q, et al. Gr-1+CD115+ immature myeloid suppressor cells mediate the development of tumor-induced T regulatory cells and T-cell anergy in tumor-bearing host. Cancer Res. 2006; 66:1123-1131.) Stimulation with Th1 cytokine IFN-γ and Th2 cytokine IL-13 induced strong expression of iNOS and Arginase 1, respectively, in ES-CD115⁺ and TD-CD115⁺ cells, but not ES-CD115⁻ cells. Taken together, these data illustrate that except for a much stronger ability to induce Treg development, ES-CD115⁺ cells are very similar to TD-CD115⁺ cells in the context of mechanisms used to mediate immunosuppressive function.

### Example 4

### ES-MDSC Sub-Populations have Distinct Phenotype and Developmental Potentials

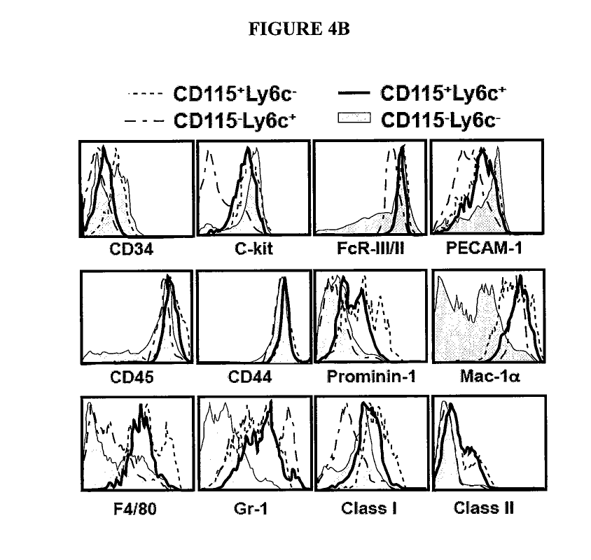

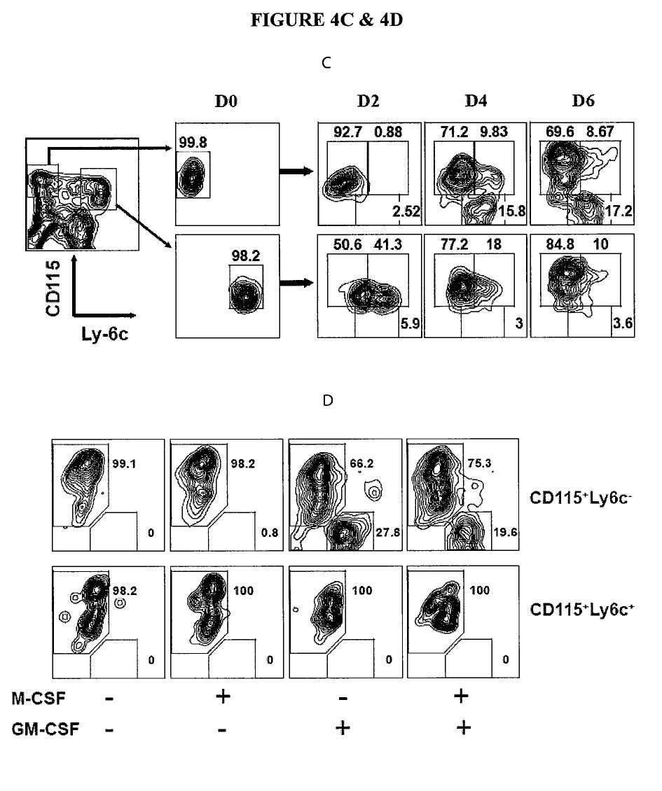

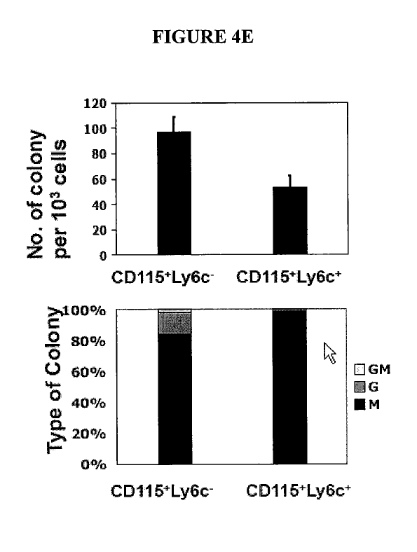

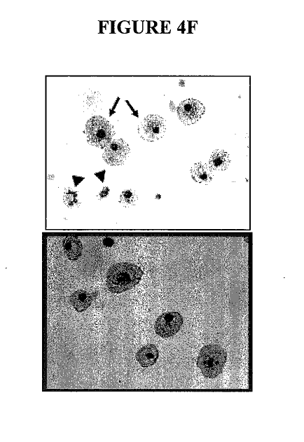

To better understand and define the unique properties of ES-MDSCs, a variety of experiments to characterize ES-MDSCs were performed. Differentiated cells were FACS-sorted, centrifuged onto slides using a cytospin, and stained by May-Giemsa (original magnification: 1000×). Morphologically, in comparison to the CD115⁻Ly-6C⁺ cells (granunolocytes, FIG. 4A, lower left) and CD115⁻Ly-6C⁻ cells (comprised of differentiated and undifferentiated cells, FIG. 4A, lower right), both CD115⁺Ly-6C⁻ (FIG. 4A, upper left) and CD115⁺Ly-6C⁺ (FIG. 4A, upper right) populations were highly homogenous with a relatively big size and a greater nuclear to cytoplasmic ratio, indicating that they are intermediately differentiated cells.

This evaluation was supported by a characteristic phenotype of these cells expressing CD34, c-Kit, FcR-III/II, and Prominin-1 (CD133) (FIG. 4B), surface antigens normally detected on hematopoietic cells at various stage of differentiation such as common myeloid progenitors (CMPs) and granulocyte/macrophage progenitors (GMPs). Although the two CD115-positive populations were not clearly distinguishable from each other in morphology, the differential expression of CD34, Prominin-1 (CD133), and the myeloid specific markers Mac-1 and Gr-1 discriminated them into distinct cell types, and suggested that the CD115⁺Ly-6C⁻ cells were at an earlier stage of differentiation.

To more precisely assess their developmental stage and lineage potentials, the FACS-sorted CD115⁺Ly-6C⁻ and CD115⁺Ly-6C⁺ cells were re-differentiated in vitro in the presence of a more complete cytokine combination. While a majority of the originally seeded CD115⁺Ly-6C⁻ (FIG. 4C, upper panel) and CD115⁺Ly-6C⁺ (FIG. 4C, lower panel) cells progressed gradually to become a population expressing CD115 and lower level of Ly-6C (these cells were immature macrophages as evaluated by Giemsa staining), the CD115⁺Ly-6C⁻ cell cultures also gave rise to two additional minor populations, CD115⁺Ly-6C⁺ cells and CD115⁻ Ly-6C⁺ (granulocyte), lineage potentials of which were entirely dependent on GM-CSF (FIG. 4D). These results not only revealed a precursor-progeny relationship between the CD115⁺Ly-6C⁻ and CD115⁺Ly-6C⁺ cells, but also suggest that they were the GMPs- and immature monocyte-like cells, respectively. The distinct developmental potentials of CD115⁺Ly-6C⁻ and CD115⁺Ly-6C⁺ cells were further confirmed by colony-forming assay. The CD115⁺Ly-6C⁻ cells had stronger colony-forming activity in methylcellulose (FIG. 4E), gave rise to colonies of macrophage (M), granulocyte (G), and occasional granulocyte/macrophage (GM) mixed colonies, whereas the CD115⁺Ly-6C⁺ cells formed only M colonies (FIG. 4E). These observations were verified by examining the morphology of colony components. Unlike the CD115⁺Ly-6C⁺ cell-derived colonies consisting solely of macrophages, colonies pooled from CD115⁺Ly-6C⁻ cell cultures contained both macrophages and granulocytes (FIG. 4F).

### Example 5

### ES-MDSCs Prevent GVHD Following Allogeneic Bone Marrow Transplantation

To examine whether ES-MDSCs would be effective in preventing allo-HSCT-associated GVHD allo-HSCT experiments were carried out. Specifically, lethally irradiated hosts (BALB/c, H-2^{d}) were left untreated or within 24 hours were adoptively transferred with 129SvEv (H-2^{b})-derived T-cell depleted bone marrow cells (TCDBM) alone or TCDBM plus purified 129SvEv spleenic T cells (T) or TCDBM plus T and ES cell-derived CD115⁺ cells (ES-MDSCs).

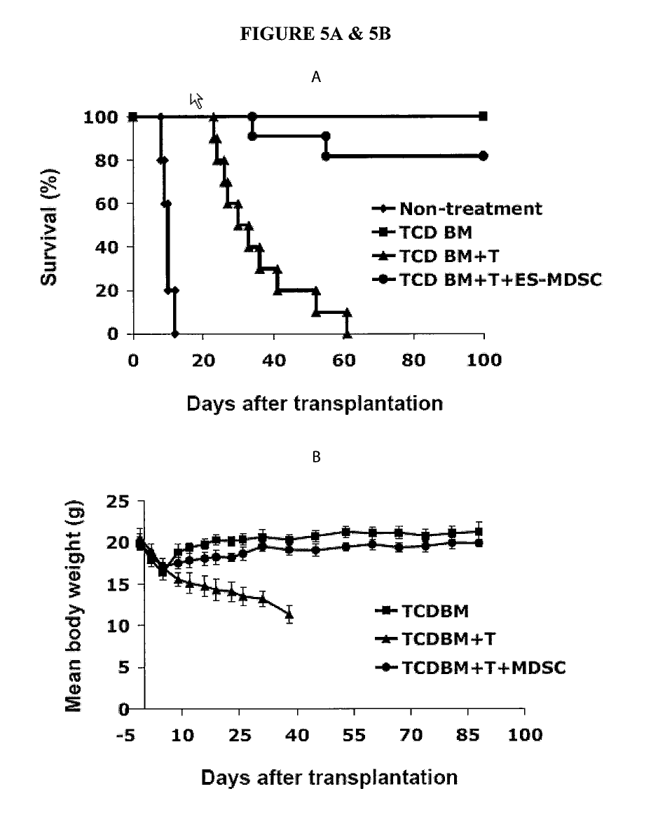

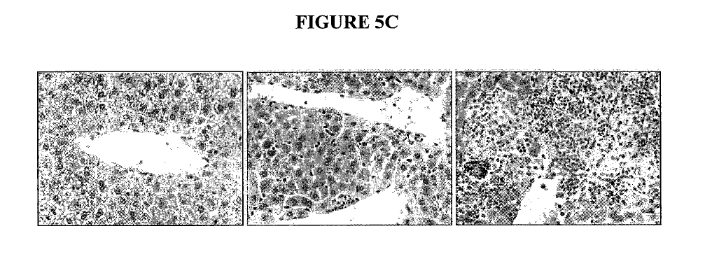

All lethally irradiated mice without treatment died within 15 days whereas mice treated with TCDBM alone were healthy and survived for the experimental period (FIG. 5A). Lethally irradiated (8 Gy, total body irradiation (“TBI”)) BALB/c mice were left untreated (♦, n=6) or transferred via tail vein injection with 129SvEv T cell-depleted bone marrow cells (TCDBM, 5×10⁶/mouse) alone (▪, n=6), or TCDBM plus purified 129SvEv splenic T cells (T, 5×10⁵/mouse) (▴, n=10), or TCDBM plus T and ES-MDSCs (2×10⁶/mouse) (, n=11). Two additional treatments of ES-MDSCs (2×10⁶/mouse, each) were given to ES-MDSC recipients on days 4 and 10 after the initial transplantation. Data shown are combined results from two independent experiments. As expected, mice that had received TCDBM plus donor-type T cells developed severe signs of GVHD (loss of hair, hunched posture, diarrhea, and weight loss) and most of animals were dead within 40 days after transplantation (FIG. 5A, B).

Strikingly, when co-grafted with ES-MDSCs along with TCDBM plus T cells, mice were largely protected from GVHD lethality and 81.8% of the animals survived for more than 100 days (P<0.0001; log-rank test). Compared to TCDBM alone treated animals, the mean body weights of the ES-MDSCs recipients were slightly lower but showed no statistically significant difference (P>0.05; student test) at all time points examined (FIG. 5B). In correspondence to their survival and body weight change, histological examination revealed dense lymphocyte infiltration in the periportal area of liver obtained from TCDBM plus T cell-treated mice (FIG. 5C, right panel), in contrast to tissue samples recovered from ES-MDSC-treated mice (FIG. 5C, middle panel) or recipients with TCDBM-only transfer (FIG. 5C, left panel). Representative micrographs are shown.

Similar histological features within respective groups were also observed in other GVHD target organs e.g. small intestine and skin. These results establish that ES-MDSCs are able to prevent the development of GVHD induced by alloreactive T cells, resulting in a tremendous improvement in animal survival after allo-HSCT.

### Example 6

### DEVELOPMENT of MDSCs from Bone Marrow Hematopoietic Stem/Progenitor Cells

The efficient derivation of MDSCs from ES cells led to examining whether the cytokine requirement identified with ES cells can be applied to generation of MDSCs from bone marrow hematopoietic/progenitor cells. Development of such a system not only helps establish an additional source for MDSCs, but also provides an alternative platform to dissect the mechanisms underlying the differentiation and accumulation of MDSCs in tumor-bearing host. The first step was depleting naïve bone marrow cells of lineage positive cells (FIG. 6A, left dot plot) and sorting them into Sca1+ and Sca1− cells by FACS (FIG. 6a, middle panel). These highly purified hematopoietic/progenitor cells were then separately subjected to differentiation as described in the Methods section (supra). BM cells isolated from B6 mice were first depleted of lineage positive cells (left plot shows after depletion) and then FACS-sorted into Sca1⁺ and Sca1⁻ cells (middle panel dot plots). Purified cells were differentiated using similar culture condition as identified in derivation of ES-MDSCs.

Surprisingly, under a similar culture condition (cytokine mix) defined with ES cells, roughly 60% of CD115⁺Ly-6C⁺ cells was induced after an 8-day culture (3-days in M1+5-days in M2) from the Sca1⁺ lin⁻ and Sca1⁻ lin⁻ fraction cultures, respectively (FIG. 6A, right panel). The marrow Sca1⁺lin⁻ cells appeared to be superior to the Sca1⁻lin⁻ fraction, as the former, in addition to generating a higher cell percentage, also enabled a sustainable production (at least 15 days in M2) of CD115⁺Ly-6C⁺ cells which were dramatically decreased in the Sca1⁻lin⁻ fraction after 6 days, possibly due to the exhaustion of the pool of CD115⁺Ly-6C⁺ progenitors. Interestingly, only the Sca1⁺Lin⁻ but not Sca1⁻Lin⁻ cells gave rise to the CD115⁺Ly-6C⁻ population, suggesting that the CD115⁺Ly-6C⁻ cells develop from progenitors of earlier stage (compared to the precusors of CD115⁺Ly-6C⁺ cells), and indirectly supporting the conclusion that the ES cell-derived CD115⁺Ly-6C⁻ and CD115⁺Ly-6C⁺ populations are GMP- and immature monocyte-like cells, respectively. In comparison to TD-MDSCs, marrow hematopoietic stem/progenitor cells-derived CD115⁺ cells (HSC-MDSCs), like the ES-MDSCs, not only displayed stronger suppressive activity against T cell proliferation stimulated by anti-CD3/anti-CD28 (FIG. 6B), but also were more efficient inducing CD4⁺CD25⁺Foxp3⁺Treg development (FIG. 6C). HS-MDSCs or TD-MDSCs were co-incubated with B6 splenocytes at different ratios in the presence of anti-CD3/anti-CD28 and [³H]-thymidine was pulsed for the final 8 hours of a 3-day culture. MDSCs were co-incubated for 5 days with B6 splenocytes at a 1:4 ratios in the presence of anti-CD3/anti-CD28 and Foxp3 expression were analyzed via intracellular staining.

To delineate which of the aforementioned suppressive mechanism(s) play a major role, the functional activity of HS-MDSCs was evaluated when specific pathway(s) were interfered. To evaluate the role of iNOS, HS-MDSCs (developed from Lin− B6 BM cells) or TD-MDSCs were co-incubated with B6 splenocytes at a 1:2 ratio in the absence or presence of various concentrations of L-NMMA (L) or NOHA (N), or combination of L-NMMA and NOHA. Cells were stimulated with anti-CD3/anti-CD28 and [3H]-thymidine was pulsed for the final 8 hours of a 3-day culture. Suppression of T cells was already substantially reversed by the iNOS inhibitor L-NMMA at a low concentration (5 μM) (*P<0.05 for HSC MDSC, **P<0.01 for TD-MDSC, versus cells without LNMMA treatment). In contrast, addition of arginase inhibitor NOHA did not interfere significantly with the activity of HSC-MDSC or TD-MDSC, even at a high concentration (500 μM) (P>0.05). Whereas NOHA (arginase 1 inhibitor) displayed minimal effects even at a high concentration (500 μM), the addition of L-NMMA (N^{G}-monomethyl-L-arginine, a specific iNOS inhibitor) partially but significantly reversed the suppression of both HS- and TD-MDSCs against T-cell proliferation in a dose-dependent manner (FIG. 6E).

The critical role of iNOS was confirmed by examining MDSCs deficient in inos (inos^{-/-}) (FIG. 6D, left panel). The role of IL-10, iNOS, and IL-4 in the suppression mediated by HS-MDSC was evalauated. HS-MDSCs developed from Lin− BM cells of indicated strains of mice were co-incubated with B6 (for il10^{-/-} and inos^{-/-} MDSCs) or BALB/c (for il-4^{-/-} MDSCs, BALB/c background) splenocytes at different ratios in the presence of anti-CD3/anti-CD28. [3H]-Thymidine was pulsed for the final 8 hours of a 3-day culture MDSCs or inos^{-/-} MDSCs vs. wild-type (wt) B6 MDSCs, *P<0.05; **P<0.01; ***P<0.001). Interestingly, MDSCs also showed significantly lower suppressive activity, though to a lesser extent compared to inos^{-/-} MDSCs. In contrast, T-cell proliferation was only slightly restored in the presence of il-4^{-/-} MDSCs (BALB/c background) as compared to wild type BALB/c HS-MDSCs (FIG. 6D, right panel). Taken together, the results suggest that IL-10 and iNOS are involved in HS-MDSC-mediated T-cell suppression.

Discussion

Methods for directed differentiation of mouse ES cells into myeloid-derived suppressor cells (MDSCs) as described herein provide the basis for differentiating any ES cells into MDSCs. Using this differentiation system it has been demonstrated that functionally active MDSCs can be generated in vitro from mouse ES cells, and these methods have also been extended to the bone marrow system, enabling MDSC production at an even higher efficiency from purified marrow hematopoietic stem/progenitor cells. The in vitro differentiation of ES or HS cells into MDSCs will provide useful tools for basic research and clinical applications.

Two homogenous populations of MDSCs were derived in these experiments from either ES or HS cell cultures: CD115⁺Ly-6C⁺ and CD115⁺Ly-6C⁻ cells. ES cell-derived CD115⁺Ly-6C⁺ cells were morphologically, phenotypically, and developmentally similar to the ex vivo BM CD115⁺Gr1⁺F4/80⁺ cells, a monocytic type that has been shown to be a major component of the MDSCs found in tumor-bearing mice. (Huang B, Pan P Y, Li Q, et al. Gr-1+CD115+ immature myeloid suppressor cells mediate the development of tumor-induced T regulatory cells and T-cell anergy in tumor-bearing host. Cancer Res. 2006; 66:1123-1131; Gallina G, Dolcetti L, Serafini P, et al. Tumors induce a subset of inflammatory monocytes with immunosuppressive activity on CD8+ T cells. J Clin Invest. 2006; 116:2777-2790.) The ES-CD115⁺Ly-6C⁻ cells, distinctively characterized by the high expression of myeloid progenitor markers (e.g. CD34, c-Kit, FcR-III/II, Prominin-1) and relatively low expression of myeloid lineage markers (e.g. Mac-1 and Gr-1), closely resembled the well-characterized GMPs as assessed by re-differentiation and colony-forming assays. This conclusion is further substantiated by the observation that a considerable potion of CD115⁺Ly-6C⁻ cells could be derived only from the marrow Sca1⁺Lin⁻ fraction (due to the existence of more primitive cell populations upstream of GMP, e.g. HSCs, multi-potential progenitors (MPPs), and/or CMPs) but not from the marrow Sca1⁻Lin⁻ fraction, cells of which themselves might be GMPs or GMP-containing population as determined by their CD115⁺Ly-6C⁺ (monocytes) and CD115⁻Ly6C⁺ (granulocytes) cell potentials. It is generally believed that MDSCs encompass immature dendritic cells, immature macrophages, monocyte, and myeloid cells at earlier stages. (Serafini P, Borrello I, Bronte V. Myeloid suppressor cells in cancer: recruitment, phenotype, properties, and mechanisms of immune suppression. Semin Cancer Biol. 2006; 16:53-65). However, a counterpart for the ES- or HS-derived CD115⁺Ly-6C⁻ cells with a suppressor phenotype has not been documented in tumor- or other pathogen-elicited MDSCs.

ES-MDSCs were highly suppressive against T cell proliferation stimulated by either polyclonal stimulus or allo-antigens in vitro. The suppressive function of ES-MDSCs may involve multiple yet similar mechanisms essential for mediating suppression of TD-MDSCs. Although no substantial difference were detected between ES-MDSCs and TD-MDSCs in the NO production, expression of arginase-1, iNOS, IL-10, and TGF-13, pair-wise comparison analysis (at least 3 times) revealed that ES-MDSCs showed consistently stronger suppressive activity than TD-MDSCs. A similar phenomenon was also observed with HS-MDSCs with at least functionally comparable, if not superior, to the ex vivo TD-MDSCs. More importantly, both ES-MDSCs and HS-MDSCs displayed significantly enhanced ability to induce Treg development compared to TD-MDSCs.

One of the major goals for these experiments was to generate MDSCs that are capable of inducing tolerance in vivo. By using a murine major histocompatibility complex (MHC)-mismatched HSCT model, the data showed clearly that adoptive transfer of bulk ES cell-derived CD115⁺ cells could efficiently protect animals from development of the allo-reactive T cell-mediated lethal GVHD, leading to nearly 82% long-term survival of treated mice. In these studies, MDSCs have been shown to be capable of suppressing GVHD with a comparable potency of ES-MDSCs, and more importantly maintaining the beneficial GVL effect and intact immunity in long-term survived mice (for CD115⁺Gr-1⁺F4/80⁺ cells). These data not only confirmed the exceptional immune tolerogenic role of MDSCs, but also suggest that in vitro-derived MDSCs, by virtue of providing a safe (tumor-cell free) and renewable cell source, have tremendous translational potential. These results provide the basis for similarly generating MDSCs using human embryonic stem cells or human cord blood CD34+ cells.

Various protocols based on either EB formation or ES-OP9 co-culture or combination of both methods have been established to direct ES differentiation cells into specific hematopoietic lineages. In this study a three-step strategy (see details in Methods) was adapted for derivation of MDSCs from the HoxB4 ES cell lines. EB formation, a process recapitulating most of the development events of early embryogenesis, is an important step in the in vitro differentiation of ES cells. Using a well-defined culture condition, ample and healthy EB colonies could be generated from HoxB4 ES cells, day 6 EBs were found to be the most effective in the M2 or M2+M-CSF medium with regard to production of CD115⁺Ly-6C⁻ cells, and thus were chosen for subsequent differentiation.

A variety of cytokines or growth factors, e.g. stem cell factor (SCF) have been shown to regulate the accumulation or migration of MDSCs in vivo. (Pan P Y, Wang G X, Yin B, et al. Reversion of immune tolerance in advanced malignancy: modulation of myeloid-derived suppressor cell development by blockade of stem-cell factor function. Blood. 2008; 111:219-228), VEGF (Melani C, Chiodoni C, Forni G, Colombo M P. Myeloid cell expansion elicited by the progression of spontaneous mammary carcinomas in c-erbB-2 transgenic BALB/c mice suppresses immune reactivity. Blood. 2003; 102:2138-2145, Gabrilovich D, Ishida T, Oyama T, et al. Vascular endothelial growth factor inhibits the development of dendritic cells and dramatically affects the differentiation of multiple hematopoietic lineages in vivo. Blood. 1998; 92:4150-4166, IL-β, GM-CSF (Young M R, Wright M A, Lozano Y, et al. Increased recurrence and metastasis in patients whose primary head and neck squamous cell carcinomas secreted granulocyte-macrophage colony-stimulating factor and contained CD34+ natural suppressor cells. Int J. Cancer. 1997; 74:69-74, Serafini P, Carbley R, Noonan K A, Tan G, Bronte V, Borrello I. High-dose granulocyte-macrophage colony-stimulating factor-producing vaccines impair the immune response through the recruitment of myeloid suppressor cells. Cancer Res. 2004; 64:6337-6343), G-CSF (J. Cell Biochem., 2006 Oct. 15; 99(3):690-705), and M-CSF (Wing E J, Magee D M, Pearson A C, Waheed A, Shadduck R K. Peritoneal macrophages exposed to purified macrophage colony-stimulating factor (M-CSF) suppress mitogen- and antigen-stimulated lymphocyte proliferation. J Immunol. 1986; 137:2768-2773, Menetrier-Caux C, Montmain G, Dieu M C, et al. Inhibition of the differentiation of dendritic cells from CD34(+) progenitors by tumor cells: role of interleukin-6 and macrophage colony-stimulating factor. Blood. 1998; 92:4778-4791). In the in vitro system as described herein, a cytokine cocktail composed of KL, VEGF, Flt3L, and TPO was identified to support the development of CD115⁺Ly-6C⁺ cells from ES or HS cells, which likely results primarily from a synergistic or additive effect of these factors, as none of them singularly was sufficient to induce the CD115⁺Ly-6C⁺ population. The further addition of M-CSF along with M2 dramatically increased the frequency of both CD115 positive populations to over 2 fold.

The bone marrow system offers another source for in vitro derivation of MDSCs. Using a similar culture condition (same cytokine mix) identified with ES cells, the data and methods described herein showed that the CD115⁺Ly-6C⁺ population was induced much more robustly from the marrow progenitors, particularly the Sca1⁺Lin⁻ fraction. While the BM or ES cell system will provide a platform for investigating the roles of cytokines and underlying pathways leading to aberrant expansion of MDSCs in vivo, will also be well suited to use to develop genetically modified MDSCs (due to relatively easier to transfect), or to generate “customized” (immunologically compatible) MDSCs (through the somatic cell nuclear transfer techniques or therapeutic cloning), which could substantially extend the applicability of MDSCs in clinical use.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

While the compositions and methods of this invention have been described in terms of specific embodiments, it will be apparent to those of skill in the art that variations may be applied to the compositions and methods and in the steps or in the sequence of steps of the method described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope of the invention as defined by the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. A method of preparing an isolated myeloid derived suppressor cell (MDSC) comprising:
a) contacting an embryonic stem (ES) cell with an effective amount of kit ligand (KL) (stem cell factor), vascular endothelial growth factor (VEGF), FMS-like tyrosine kinase 3 (Flt3L), thrombopoietin (TPO), and macrophage colony-stimulating factor (M-CSF); and
b) culturing said ES cell under conditions suitable for propagation of said cell, thereby obtaining a preparation of an isolated MDSC.

2. The method of claim 1, further comprising cryopreservation of said MDSC.

3. The method of claim 1, wherein the ES cell is a mammalian ES cell.

4. The method of claim 3, wherein the ES cell is a human ES cell.

5. The method of claim 1, wherein the isolated MDSC expresses at least one of the cell surface markers selected from the group consisting of CD33, CD115, F4/80, Ly-6C, CD11b, Gr-1, and IL-4R.

6. An isolated MDSC obtained by the method according to claim 1.

7. A method of preparing an isolated myeloid derived suppressor cell (MDSC) comprising:
a) contacting a hematopoietic stem cell (HSC) with an effective amount of kit ligand (KL) (stem cell factor), vascular endothelial growth factor (VEGF), FMS-like tyrosine kinase 3 (Flt3L), thrombopoietin (TPO), and macrophage colony-stimulating factor (M-CSF); and
b) culturing said HSC under conditions suitable for propagation of said cell, thereby obtaining a preparation of an isolated MDSC.

8. The method of claim 7, further comprising cryopreservation of said MDSC.

9. The method of claim 7, wherein the HSC is a mammalian HSC.

10. The method of claim 9, wherein the HSC is a human HSC.

11. The method of claim 7, wherein the isolated MDSC expresses at least one of the cell surface markers selected from the group consisting CD33, CD115, F4/80, Ly-6C, CD11b, Gr-1, and IL-4R.

12. An isolated MDSC obtained by the method according to claim 7.

13. A method of treating a disorder amenable to cell-based treatment in a mammal, comprising administering a pharmaceutically effective amount of the isolated MDSC of claim 6 to a mammal in need thereof.

14. The method of claim 13, wherein the disorder is selected from the group consisting of graft-versus-host disease (GVHD) and an autoimmune disorder.

15. The method of claim 3, wherein the isolated MDSC expresses at least one of the cell surface markers selected from the group consisting of CD115, F4/80, Ly-6C, CD11b, Gr-1, VEGF receptor, CD40 and IL-4R.

16. The method of claim 4, wherein the isolated MDSC expresses at least one of the cell surface markers selected from the group consisting of CD11b, CD33, CD15, and CD16.

17. The method of claim 4, wherein the isolated MDSC expresses CD11b and CD33.

18. The method of claim 3, wherein the isolated MDSC expresses CD11b and Gr-1.

19. The method of claim 17, wherein the isolated MDSC further expresses CD15 or CD16.

20. The method of claim 17, wherein the isolated MDSC further expresses CD15 and CD16.

21. A method of treating a disorder amenable to cell-based treatment in a mammal, comprising administering a pharmaceutically effective amount of the isolated MDSC of claim 12 to a mammal in need thereof.

22. The method of claim 21, wherein the disorder is selected from the group consisting of graft-versus-host disease (GVHD) and an autoimmune disorder.

23. The method of claim 9, wherein the isolated MDSC expresses at least one of the cell surface markers selected from the group consisting of CD115, F4/80, Ly-6C, CD11b, Gr-1, VEGF receptor, CD40 and IL-4R.

24. The method of claim 10, wherein the isolated MDSC expresses at least one of the cell surface markers selected from the group consisting of CD11b, CD33, CD15, and CD16.

25. The method of claim 10, wherein the isolated MDSC expresses CD11b and CD33.

26. The method of claim 9, wherein the isolated MDSC expresses CD11b and Gr-1.

27. The method of claim 25, wherein the isolated MDSC further expresses CD15 or CD16.

28. The method of claim 25, wherein the isolated MDSC further expresses CD15 and CD16.


SEQUENCE LISTING (14 sequences)

SEQ ID NO: 1 - DNA, 2973 bp, Homo sapiens
agtgcagcgcagaagacagagggtgactaggaagacgcgcgagcggggctggccggccggcgggtgggggaggggaggcgggggaaggcggctgagtgggcctctggagtgtgtgtgtctgtgtcagtgtgtgtgtgtgtgtgtgtatgtgtgtgtctggcgcctggccagggtgatttcccataaaccacatgccccccagtcctctcttaaaaggctgtgccgagggctggccagtgaggctcggcccggggaaagtgaaagtttgcctgggtcctctcggcgccagagccgctctccgcatcccaggacagcggtgcggccctcggccggggcgcccactccgcagcagccagcgagcgagcgagcgagcgagggcggccgacgcgcccggccgggacccagctgcccgtatgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacacccccaatgccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactggtaccgccttgcccagggtgggccccgtgaggcccactggccaggactggaatcacaccccccagaagacagaccatccatctgccctgctcagagaccccccggagccaggctctcccaggatctcatcactgcgcccccagggcctcagcaacccctccaccctctctgctcagccacagctttccagaagccactcctcgggcagcgtgctgccccttggggagctggagggcaggaggagcaccagggatcggaggagccccgcagagccagaaggaggaccagcaagtgaaggggcagccaggcccctgccccgttttaactccgttcctttgactgacacaggccatgagaggcagtccgagggatcctccagcccgcagctccaggagtctgtcttccacctgctggtgcccagtgtcatcctggtcttgctggccgtcggaggcctcttgttctacaggtggaggcggcggagccatcaagagcctcagagagcggattctcccttggagcaaccagagggcagccccctgactcaggatgacagacaggtggaactgccagtgtagagggaattctaagctggacgcacagaacagtctctccgtgggaggagacattatggggcgtccaccaccacccctccctggccatcctcctggaatgtggtctgccctccaccagagctcctgcctgccaggactggaccagagcagccaggctggggcccctctgtctcaacccgcagacccttgactgaatgagagaggccagaggatgctccccatgctgccactatttattgtgagccctggaggctcccatgtgcttgaggaaggctggtgagcccggctcaggaccctcttccctcaggggctgcaccctcctctcactcccttccatgccggaacccaggccagggacccaccggcctgtggtttgtgggaaagcagggtggacgctgaggagtgaaagaaccctgcacccagagggcctgcctggtgccaaggtatcccagcctggacaggcatggacctgtctccagagagaggagcctgaagttcgtggggcgggacagcgtcggcctgatttcccgtaaaggtgtgcagcctgagagacgggaagaggaggcctctggacctgctggtctgcactgacagcctgaagggtctacaccctcggctcacctaagtgccctgtgctggttgccaggcgcagaggggaggccagccctgccctcaggacctgcctgacctgccagtgatgccaagagggggatcaagcactggcctctgcccctcctccttccagcacctgccagagcttctccaggaggccaagcagaggctcccctcatgaaggaagccattgcactgtgaacactgtacctgcctgctgaacagcctgcccccgtccatccatgagccagcatccgtccgtcctccactctccagcctctcccca

SEQ ID NO: 2 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSSSPQLQESVFHLLVPSVILVLLAVGGLLFYRWRRRSHQEPQRADSPLEQPEGSPLTQDDRQVELPV

SEQ ID NO: 3 - DNA, 4192 bp, Mus musculus
acagagcgacggggaagagagctagcggggacgaccaggcggcccgcttgggggaagggagtcggcggctcagtgggcctctggggtgtagtatgtgtcagtgcctgtgagtgtgtttgtgtgtgtgtatgtctgtgtgtgtctggcggagagccagggtgatttcccataaaccacatgccccgccagcccgcccgcttaaaaggctgtgccgagggctggccagcgaagctcggccaggggaaagtgaaagtttgcctcggtgctctcggtgtcgctgcggctctctgcatcccaggacagcggcgtggccctcgaccggggcgcgggctcttcagccactagcgagcaagggagcgagcgaaccagggcggccaacacgccgtgccgggacccagctgcccgtatgaccgcgcggggcgccgcggggcgctgcccttcttcgacatggctgggctcccggctgctgctggtctgtctcctcatgagcaggagtattgccaaggaggtgtcagaacactgtagccacatgattgggaatggacacctgaaggtcctgcagcagttgatcgacagtcaaatggagacttcatgccagattgcctttgaatttgtagaccaggaacagctggatgatcctgtttgctacctaaagaaggccttttttctggtacaagacataatagatgagaccatgcgctttaaagacaacacccccaatgctaacgccaccgagaggctccaggaactctccaataacctgaacagctgcttcaccaaggactatgaggagcagaacaaggcctgtgtccgaactttccatgagactcctctccagctgctggagaagatcaagaacttctttaatgaaacaaagaatctccttgaaaaggactggaacatttttaccaagaactgcaacaacagctttgctaagtgctctagccgagatgtggtgaccaagcctgattgcaactgcctgtaccctaaagccacccctagcagtgacccggcctctgcctcccctcaccagccccccgccccctccatggcccctctggctggcttggcttgggatgattctcagaggacagagggcagctccctcttgcccagtgagcttccccttcgcatagaggacccaggcagtgccaagcagcgaccacccaggagtacctgccagaccctcgagtcaacagagcaaccaaaccatggggacagactcactgaggactcacaacctcatccttctgcgggggggcccgtccctggggtggaagacattcttgaatcttcactgggcactaactgggtcctagaagaagcttctggagaggctagtgagggatttttgacccaggaagcaaagttttccccctccacgcctgtagggggcagcatccaggcagagactgacagacccagggccctctcagcatctccattccctaaatcaacagaggaccaaaagccagtggatataacagacaggccgttgacagaggtgaaccctatgagacccattggccagacacagaataatactcctgagaagactgatggtacatccacgctgcgtgaagaccaccaggagccaggctctccccatattgcgacaccgaatccccaacgagtcagcaactcagccacccccgttgctcagttactgcttcccaaaagccactcttggggcattgtgctgccccttggggagcttgagggcaagagaagtaccagggatcgaaggagccccgcagagctggaaggaggatcagcaagtgagggggcagccaggcctgtggcccgttttaattccattcctttgactgacacaggccatgtggagcagcatgagggatcctctgacccccagatccctgagtctgtcttccacctgctggtgccgggcatcatcctagtcttgctgactgttgggggcctcctgttctacaagtggaagtggaggagccatcgagaccctcagacattggattcttctgtggggcgaccagaggacagctccctgacccaggatgaggacagacaggtggaactgccagtatagaaaggattctatgctgggcacacaggactatctctttatggaaggagacatatgggaacatccaccactaccctctcctaccatcttcctgggaatgtggcctaccactaccagagctcctgcctaccaagactggatgaaagaagcagctttgatggggtctttccatcctcacccttagactctcaaccaaagagaaagggctggaggatgccccccacatactgccactatttattgtgggccctggaggctccctgcattggaggaagggcagctcagcagctcaggaccctttcccttaggggctgcttcctcccctcaaaaccagaacctggcaagggactcactagcctggatggcccatgggagaccaggacagatgagaaggagcagaagagccctgtgcccagaagacccaactggtgccaaggaatcccagcatggacaggcagggacctgtttcccaagaagagagcctgatattcaaagggtgggacagcatctgcccgacttcccgtaaaggcataaaggcacgcagcccaaaagacgggaagaggaggcctttggctgcttgtgttgacagcttaaaggggtctacaccctcaacttgcttaagtgccctctgctgatagccaggaaggagggagaccagccctgcccctcaggacctgacctggctcatgatgccaagaggaagacagagctctagcctcgtcttctcctgcccacagcccctgccagagttcttttgcccagcagaggcacccctcatgaaggaagccattgcactgtgaatactgaacctgcctgctgaacagcctgtcccatccatccctatgagtgaccatccgtccgaatgttctcccacttccttcagcctctcctcggcttcttgcactgagctggcctcacgtgttgactgagggagcccctgagccccaaccttcccctgcctcagcctttgattgtccagggtgaagctgtgggagaaccgcctgggctaccagtcagagctggtctttgggctgtgttccttgcccaggtttctgcatcttgcactttgacattcccaggagggaagtgactagtggaagggagagaggaaggggaggcagagacaaaggccacaggcagagctatgaatgagaatgggtcttgaaaatatgtgtgcacccctaagcttgaaattgatctctatactctagcccctcagccagcctccttcctgttgtctgaaacctggagctaagcaggttgtcctgtcacaagctctggggactgagctccatgctccaaccccaccctcttctgacctttgttctccagacctgacccaggtaggcaagggtaccctcccagtctcacctaccatactgtgccatctctagccaagcaagccaggtttagagaagggtcaaaaaaaaaaaaaagggttgtttacttccaacttgttctgatgccctctgtttcccaggccaggcttgtctgtggtgacctgggcatgggtgacagggctctcatttgccccttggtctctttatgctgctgagtccccctttcctgccctccctggctactgggtcaataatctttcaggccatgaatctgggaggagagtggtctgtaagctccatcagccctgtcctgagacagcaggggggaaggacactggagactttcttgtggggcttacttagccttctggttacagactatttccatgctagaaaatacatattttaaaatagaaggaaaaacacagaaacaaaacaaaacaaggcattctctacccctccaccttaaacatatattattaaagacagaagagaaaatccaacccattgcaagaagctctttgtgggtgcctggttacatcggagcaggggagcctcaaatccacctttggagccgcccctgtgtgcattaggaacccttctctcctctgagaaagctcagagggagcactgcctcacaaactgtgagactgcgttttttatacttggaagtggtgaattattttatataaggtcatttaaatatctatttaaaaaataggaagctgcttttatatttaataataaaagaagtgcacaagctgccacgtgtgaaaaaaaaaaaaaaaaa

SEQ ID NO: 4 - protein, 552 aa, Mus musculus
MTARGAAGRCPSSTWLGSRLLLVCLLMSRSIAKEVSEHCSHMIGNGHLKVLQQLIDSQMETSCQIAFEFVDQEQLDDPVCYLKKAFFLVQDIIDETMRFKDNTPNANATERLQELSNNLNSCFTKDYEEQNKACVRTFHETPLQLLEKIKNFFNETKNLLEKDWNIFTKNCNNSFAKCSSRDVVTKPDCNCLYPKATPSSDPASASPHQPPAPSMAPLAGLAWDDSQRTEGSSLLPSELPLRIEDPGSAKQRPPRSTCQTLESTEQPNHGDRLTEDSQPHPSAGGPVPGVEDILESSLGTNWVLEEASGEASEGFLTQEAKFSPSTPVGGSIQAETDRPRALSASPFPKSTEDQKPVDITDRPLTEVNPMRPIGQTQNNTPEKTDGTSTLREDHQEPGSPHIATPNPQRVSNSATPVAQLLLPKSHSWGIVLPLGELEGKRSTRDRRSPAELEGGSASEGAARPVARFNSIPLTDTGHVEQHEGSSDPQIPESVFHLLVPGIILVLLTVGGLLFYKWKWRSHRDPQTLDSSVGRPEDSSLTQDEDRQVELPV

SEQ ID NO: 5 - DNA, 25 bp, Artificial Sequence
  Primer
gagattggagttcgagacttctgtg

SEQ ID NO: 6 - DNA, 20 bp, Artificial Sequence
  Primer
tggctagtgcttcagacttc

SEQ ID NO: 7 - DNA, 23 bp, Artificial Sequence
  Primer
cagagtatgacgtgagagaccac

SEQ ID NO: 8 - DNA, 25 bp, Artificial Sequence
  Primer
cagcttgtctacttcagtcatggag

SEQ ID NO: 9 - DNA, 21 bp, Artificial Sequence
  Primer
ctcttactgactggcatgagg

SEQ ID NO: 10 - DNA, 25 bp, Artificial Sequence
  Primer
ccttgtagacaccttggtcttggag

SEQ ID NO: 11 - DNA, 22 bp, Artificial Sequence
  Primer
gtggtatactgagacaccttgg

SEQ ID NO: 12 - DNA, 22 bp, Artificial Sequence
  Primer
ccttagtttggacaggatctgg

SEQ ID NO: 13 - DNA, 22 bp, Artificial Sequence
  Primer
gtggagattgttgccatcaacg

SEQ ID NO: 14 - DNA, 25 bp, Artificial Sequence
  Primer
cagtggatgcagggatgatgttctg